(19) United States
(12) Patent Application Publication
(10) Pub. No.: US 2011/0158988 A1
(43) Pub. Date: Jun. 30, 2011

(54) ANTIBODIES AGAINST EXTRACELLULAR DOMAINS 2 AND 3 OR HER2

(75) Inventors: Mathias Uhlen, Stocksund (SE); Johan Rockberg, Stockholm (SE)

(73) Assignee: ATLAS THERAPEUTICS AB, Stockholm (SE)

(21) Appl. No.: 12/997,823

(22) PCT Filed: Dec. 12, 2008
(86) PCT No.: PCT/SE08/00694

§ 371 Date: Mar. 11, 2011

Related U.S. Application Data
(60) Provisional application No. 61/076,352, filed on Jun. 27, 2008.

(30) Foreign Application Priority Data
Jun. 13, 2008 (JP) 2008-155676

(51) Int. Cl. A61K 39/395; C07K 16/40; G01N 33/53; C12N 15/06; C12P 21/02; A61P 35/00; A61P 35/04

(52) U.S. Cl. 424/133.1; 530/387.7; 530/387.3; 424/138.1; 436/501; 435/449; 435/70.2

(57) ABSTRACT

The present invention relates to an affinity ligand capable of selective interaction with a subset consisting of 37 consecutive amino acid residues or less from extracellular domains 2 and 3 of HER2, wherein the subset comprises the amino acid sequence LQVF and/or ESFDGD1 and to polypeptides consisting of such subsets.

### FIELD OF THE INVENTION

The present invention generally relates to amino acid subsets of the extracellular domain of HER2, antibodies targeting such subsets and related methods and uses, such as therapeutic methods and uses.

### BACKGROUND

### HER2

The human epidermal growth factor receptor 2 (HER2 or erbB-2) is a member of the epidermal growth factor receptor family (EGFR, HER2, HER3 and HER4) a family of transmembrane receptor tyrosine kinases. These receptors have an over all sequence identity of between 40-50% and have similar domains. They all contain an extracellular ligand-binding domain, a single, transmembrane spanning domain, and intracellular tyrosine kinase and regulatory domains.

### HER2 Related Disorders

Members of the epidermal growth factor receptor family have been found to promote tumor cell proliferation in a variety of cancer types, such as epithelial malignancies. HER2 has been studied in a variety of human carcinomas and have been found to be upregulated in a number of tumors, in particular in breast, lung, pancreatic and colorectal cancer and Wilm's tumor, but also in ovarian, bladder, endometrial, renal, head and neck, gastric, esophageal and prostate cancer (Ménard et al (2001) Annals of Oncology 12 (Suppl. 1) S15-S19).

### Cancer

Cancer is one of the most common causes of disease and death in the western world. In general, incidence rates increase with age for most forms of cancer. As human populations continue to live longer, due to an increase of the general health status, cancer may affect an increasing number of individuals. The cause of most common cancer types is still largely unknown, although there is an increasing body of knowledge providing a link between environmental factors (dietary, tobacco smoke, UV radiation etc) as well as genetic factors (germ line mutations in “cancer genes” such as p53, APC, BRCA1, XP etc) and the risk for development of cancer.

No definition of cancer is entirely satisfactory from a cell biological point of view, despite the fact that cancer is essentially a cellular disease and defined as a transformed cell population with net cell growth and anti-social behavior. Malignant transformation represents the transition to a malignant phenotype based on irreversible genetic alterations. Although this has not been formally proven, malignant transformation is believed to take place in one cell, from which a subsequently developed tumor originates (the “clonality of cancer” dogma). Carcinogenesis is the process by which cancer is generated and is generally accepted to include multiple events that ultimately lead to growth of a malignant tumor. This multi-step process includes several rate-limiting steps, such as addition of mutations and possibly also epigenetic events, leading to formation of cancer following stages of precancerous proliferation. The stepwise changes involve accumulation of errors (mutations) in vital regulatory pathways that determine cell division, asocial behavior and cell death. Each of these changes may provide a selective Darwinian growth advantage compared to surrounding cells, resulting in a net growth of the tumor cell population. A malignant tumor does not only necessarily consist of the transformed tumor cells themselves but also surrounding normal cells which act as a supportive stroma. This recruited cancer stroma consists of connective tissue, blood vessels and various other normal cells, e.g., inflammatory cells, which act in concert to supply the transformed tumor cells with signals necessary for continued tumor growth.

The most common forms of cancer arise in somatic cells and are predominantly of epithelial origin, e.g., prostate, breast, colon, urothelial and skin, followed by cancers originating from the hematopoetic lineage, e.g., leukemia and lymphoma, neuroectoderm, e.g., malignant gliomas, and soft tissue tumors, e.g., sarcomas.

### Breast Cancer

Breast cancer is the second most common form of cancer worldwide and by far the most frequent cancer of women. Data from the GLOBOCAM 2002 database presented by Parkin et al. reveal 1.15 million new cases in 2002 and 0.41 million deaths during the same period (Parkin D M et al. (2005) CA Cancer J Clin 55, 74-108). If detected at an early stage, the prognosis is relatively good for a patient living in a developed country, with a general five-year survival rate of 73%, compared to 57% in a developing country. The incidence is slowly increasing and about one in every nine women in the developed world is believed to get breast cancer in her lifetime. Although lifestyle changes related to female steroid hormones, including exposure to exogenous hormones, affect the risk of developing breast cancer, these factors only make up for a small fraction of the etiology, and the benefit of preventive manipulation is believed to be low. The decreased mortality is due to earlier detection by mammography screening and the use of modern adjuvant systemic treatment.

### Cancer Treatment and Therapy

Cancer treatments include, for example, surgery, radiation therapy, chemotherapy, targeted therapies, immunotherapy, hormonal therapy and angiogenesis inhibitors.

An example of a targeted therapy is treatment with therapeutic antibodies (antibody therapy), which may be an attractive approach as it targets tumor cells, in contrast to e.g. selective estrogen receptor modulators (SERMs) and chemotherapies which are normally systemic treatments.

### Treatment of Breast Cancer

Since its introduction in the late seventies, breast-conserving therapy, combining breast conserving surgery and postoperative radiotherapy, has become the primary treatment of choice in women where radical removal of the tumor can be combined with a good cosmetic result. Mastectomy is still preferable in some patients, i.e., women with small breasts, large tumors (>4 cm) or multifocal/multicentric disease.

Axillary dissection is primarily performed for diagnostic purposes and removal of at least 10 lymph nodes gives a good staging guidance with 97-98% sensitivity (Axelsson C K et al. (1992) Eur J Cancer 28A:1415-8; Recht A and Houlihan M J (1995) Cancer 6(9):1491-1512). However, the next step towards minimal surgery in the treatment of primary cancer has been the introduction of the sentinel node biopsy technique with mapping of axillary lymph nodes instead of axillary lymph node clearance, which is associated with a high complication rate. This technique was introduced as a consequence of the knowledge that most of the lymphatic drainage to the axilla from the breast initially passes through one (or a few) lymph node(s)—the sentinel node(s)—supporting that analysis of this lymph node may be a sufficient indicator of axillary node status (Veronesi U et al. (2003) New Engl J Med 349(6): 546-53.)

The concept of breast cancer as a systemic disease, i.e., the presence of disseminating micro-metastases at the time of diagnosis that may explain treatment failure after locoregional therapy, paved the way for adjuvant randomized trials in the 1970s, including endocrine therapy and chemotherapy. Adjuvant polychemotherapy has often been the standard treatment for hormone-receptor negative patients with high risk of recurrence, irrespective of nodal status. A beneficial effect on both overall- and relapse-free survival has been demonstrated, especially in premenopausal patients (EBCTCG (1998) Lancet 352(9132): 930-42). For patients with hormone-responsive disease, i.e., estrogen receptor (ER) and/or progesterone receptor (PR) positive disease, adjuvant polychemotherapy has been delivered in combination with endocrine therapy as sequential chemo-endocrine therapy. Also, adjuvant chemotherapy generally induces amenorrhea, causing a secondary endocrine effect in addition to the cytotoxic (Pagani O et al. (1998) Eur J Cancer 34(5):632-40).

Endocrine therapy is recommended for patients with hormone receptor positive tumors irrespective of age, stage and menopausal status.

In hormone-responsive premenopausal patients, ovarian ablation by surgery or irradiation, or ovarian suppression by LHRH agonists have been shown to be efficient adjuvant treatment modalities (Emens L A and Davidson N A (2003) Clin Ca Res (1 Pt 2): 468S-94S). In postmenopausal patients, ovarian ablation has no place, since the primary source of estrogen is not from ovarian synthesis but from the conversion of androstenedione to estrone and estradiol in peripheral tissues including the breast.

Tamoxifen is a selective estrogen receptor modulator (SERM) with an agonistic effect on the ER, making it a suitable treatment for advanced breast cancer in both pre- and postmenopausal women. Five years of tamoxifen as adjuvant treatment after primary surgery clearly reduces the breast cancer mortality in patients with ER positive (ER+) tumors, irrespective of lymph node status (EBCTCG (1998) Lancet 351(9114):1451-67). While tamoxifen has a protective effect against cardiovascular disease, the risk of developing endometrial cancer is increased, due to an agonistic effect on the ER in the endometrium (EBCTCG (2005) Lancet 365(9472):1687-717)

Aromatase inhibitors (AIs) function by inhibiting aromatase, the enzyme converting androgens into estrogens. AIs are not suitable for treatment of premenopausal women, as it stimulates the ovaries to an increased androgen production through the hypothalamus and pituitary gland. AIs can be given as adjuvant treatment to postmenopausal women, either alone or following tamoxifen treatment and they have been shown to significantly reduce mortality, possibly even more if given alone (Howell A et al. (1995) Lancet 345(8941):29-30; Ellis M J and Rigden C E (2006) Curr Med Res Opin 22(12):2479-87; Coates A S et al. (2007) J Clin Oncol 25(5):486-92). However, this therapy is relatively new and the long-term side effects are not yet fully known (Buzdar A et al. (2006) Lancet Oncol 7(8):633-43), but the most important are cardiovascular complications and osteoporosis.

Newly developed pure anti-estrogens such as fulvestrant, which completely blocks the ER, are currently only used in advanced breast cancer and not in the adjuvant setting (Rutqvist L E (2004) Best Pract Res Clin Endocrinol Metab 18(1): 81-95).

Adjuvant endocrine therapy has no place in hormone receptor negative breast cancer, although some studies indicate that some ER negative (ER−), i.e., ERα negative (ERα−), tumors respond to tamoxifen treatment (EBCTCG (1998) Lancet 351:1451-1467).

The Her2 gene is overexpressed in about 20% of all, and in up to 70% of lowly differentiated, breast cancers (Berger M S et al. (1988) Cancer Res 48(5):1238-43; Borg Å et al. (1990) Cancer Res 50(14): 4332-7). HER2 status may be assessed routinely, primarily by immunohistochemistry (IHC) and in cases with moderate expression, gene amplification status may be determined by fluorescence in situ hybridization (FISH) analysis. HER2 overexpression or gene amplification is commonly associated with a poor prognosis. Further, experimental data in support of a relationship between HER2 overexpression and resistance to endocrine treatment have been presented (Shou J et al. (2004) J Natl Cancer Inst 96(12):926-35). However, clinical data are not consistent (Borg Å et al. (1994) Cancer Lett 81(2):137-44, De Placido S et al. (2003) Clin Ca Res 9(3):1039-46, Rydén L et al. (2005) J Clin Oncol 23(21):4695-704).

Breast cancer is a truly heterogeneous disease and despite the increasing understanding of its nature, the available treatment options are still not fully satisfying.

### DISCLOSURE OF THE INVENTION

It is an object of an aspect of the present disclosure to provide subsets of amino acid residues from the extracellular domain of HER2. Objects of other aspects of the present disclosure are to provide affinity ligands capable of interaction with the subsets and compositions comprising such affinity ligands. Further objects of certain other aspects of the present disclosure are to provide uses and methods utilizing the subsets as targets, e.g. for therapeutic purposes.

The present invention is defined by the appending claims.

Thus, as a first aspect of the present disclosure, there is provided an affinity ligand capable of selective interaction with a subset of 37 consecutive amino acid residues or less from extracellular domains 2 and 3 of HER2 (SEQ ID NO:7), said subset comprising the amino acid sequence LQVF (SEQ ID NO:8) and/or ESFDGD (SEQ ID NO:9).

In the context of the present disclosure, “extracellular domains 2 and 3 of HER2” refers to the part of the HER2 sequence which consists of the amino acid residues of SEQ ID NO:7.

Further, in the context of the present disclosure, “consecutive amino acid residues from extracellular domains 2 and 3 of HER2” refers to a continuous part of the amino acid sequence of SEQ ID NO:7.

Also, in the context of the present disclosure, “selective interaction with a subset of amino acid residues” refers to selective interaction with the amino acid residues contained in the subset. For example, an affinity ligand capable of selective interaction with a subset of amino acid residues may be capable of selective interaction with a fragment consisting of the amino acid residues of the subset, which fragment may be present free in solution or immobilized, e.g. bound to a bead. Also, such fragment may be bound to reporter moieties for detection of interaction. As another example, “affinity ligand capable of selective interaction with a subset of amino acid residues” may refer to the case wherein the subset is comprised in a longer polypeptide, provided that it is established that the affinity ligand interacts with the amino acid residues of the subset and not the surrounding amino acid residues.

In the context of the present disclosure, “specific” or “selective” interaction of e.g., an affinity ligand with its target or antigen means that the interaction is such that a distinction between specific and non-specific, or between selective and non-selective, interaction becomes meaningful. The interaction between two proteins is sometimes measured by the dissociation constant. The dissociation constant describes the strength of binding (or affinity) between two molecules. Typically the dissociation constant between an antibody and its antigen is from 10⁻⁷ to 10⁻¹¹ M. However, high specificity does not necessarily require high affinity. Molecules with low affinity (in the molar range) for its counterpart have been shown to be as specific as molecules with much higher affinity. In the case of the present disclosure, a specific or selective interaction refers to the extent to which a particular method can be used to determine the presence and/or amount of a specific protein, the target protein or a fragment thereof, under given conditions in the presence of other proteins in a tissue sample or fluid sample of a naturally occurring or processed biological fluid. In other words, specificity or selectivity is the capacity to distinguish between related proteins. Specific and selective are sometimes used interchangeably in the present description. For example, the specificity or selectivity of an antibody may be determined as in Examples, section 4, below, wherein analysis is performed using a protein array set-up, a suspension bead array and a multiplexed competition assay, respectively. Specificity and selectivity determinations are also described in Nilsson P et al. (2005) Proteomics 5:4327-4337.

This first aspect of the present invention is based on, but not limited to, that the inventors have found that affinity ligands binding to polypeptides consisting of amino acid sequences within extracellular domains 2 and 3 of HER2, in particular polypeptides comprising the sequences LQVF (SEQ ID NO:8) and/or ESFDGD (SEQ ID NO:9), have a growth inhibiting effect on human breast cancer cells.

A polypeptide fragment of 26 amino acid residues (SEQ ID NO:16), and shorter fragments, such as a fragment of 21 amino acid residues (SEQ ID NO:19), a fragment of 12 amino acid residues (SEQ ID NO:20), two fragments of 9 amino acid residues (SEQ ID NO:15 and 18) and two fragments of 8 amino acid residues (SEQ ID NO:11 and 17), have been found to interact with antibodies exhibiting a growth inhibiting effect (see also FIG. 6).

Accordingly, in embodiments of the first aspect, the subset may consist of 30 amino acid residues or less, such as 26 amino acid residues or less. Further, the subset may for example consist of 21 amino acid residues or less, such as 16 amino acid residues or less, such as 12 amino acid residues or less, such as 9 amino acid residues or less, such as 8 amino acid residues or less.

A sufficient interaction between the affinity ligand and the subset may in some cases require amino acid sequences of various lengths. Accordingly, in embodiments of the first aspect, the subset may consist of 6 amino acid residues or more, such as 8 amino acid residues or more, such as 10 amino acid residues or more.

As shown in Examples below, an immunization yielding antibodies having a growth inhibiting effect was performed using an antigen (SEQ ID NO:1) in which the last four amino acid residues at the C-terminal end were LQVF. Accordingly, in embodiments of the first aspect, if the subset comprises the sequence LQVF, it may have 2 amino acid residues or less on the C-terminal side of LQVF, such as no amino acid residues on the C-terminal side of LQVF. That is, in some embodiments, the C-terminal of the subset may consist of . . . LQVFET (two amino acid residues on the C-terminal side of LQVF), . . . LQVFE (one amino acid residue on the C-terminal side of LQVF) or . . . LQVF (no amino acid residues on the C-terminal side of LQVF).

A number of fragments (SEQ ID NO:11 and 15-20) have been found to interact with antibodies exhibiting a growth inhibiting effect, i.e. msAb-C (see Examples, section 3). Accordingly, in embodiments of the first aspect, said subset may be selected from the group consisting of SEQ ID NO:11 and 15-20.

In further embodiments of the first aspect, the subset may comprise the sequence LQVF. In such embodiments, the subset may be selected from the group consisting of SEQ ID NO:16 and 20. SEQ ID NO:16 and 20, respectively, comprises LQVF.

In other embodiments of the first aspect, the subset may comprise the sequence ESFDGD. In such embodiments, the subset may be selected from the group consisting of SEQ ID NO:11 and 15-19. Each of SEQ ID NO:11 and 15-19 comprise ESFDGD.

In embodiments of the first aspect, the subset may comprise the sequence PESFDGD (SEQ ID NO:10) or LPESFDGD (SEQ ID NO:11).

Further, in embodiments of the first aspect, the subset may comprise the sequence ESFDGDP, such as PESFDGDP, such as LPESFDGDP.

In embodiments of the first aspect, the subset may be the sequence of amino acid residues 1-37 of SEQ ID NO:6.

As further explained in Examples, section 5, below, the inventors have found that affinity ligands of the present disclosure may inhibit growth of breast cancer cells. Accordingly, in embodiments of the first aspect, the affinity ligand may inhibit growth of human breast cancer cells, such as human breast cancer cells in culture. For example, the affinity ligand may inhibit growth of human breast cancer cells in culture by 20-100%, such as by 30-100%, relative to an affinity ligand not capable of selective interaction with the extracellular domain of HER2, such as an antibody capable of selective interaction with the intracellular domain of HER2. It is within the capabilities of those skilled in the art to perform measurements yielding such a relative growth inhibition value and to adapt such measurements to a specific case.

As an example, the measurement of a relative inhibition may be performed by adding a certain concentration of the affinity ligand to a first culture of human breast cancer cells, such as BT474 breast cancer cells, and the same concentration of an antibody capable of selective interaction with the intracellular domain of HER2, such as HPA001383 (Atlas Antibodies, Sweden), to a second culture of the same type of human breast cancer cells. After a certain time of incubation, such as 4 days, the number of cells in the respective culture is counted. The number of cells in the second culture is considered as the reference, and the growth inhibition value of the affinity ligand is calculated relative that reference. That is, if the reference is 100 cells and the first culture contained 70 cells, the growth inhibition is (100-70)/100=30%. See also Examples, Section 5, below.

Accordingly, the human breast cancer cells may for example be BT474 breast cancer cells. And further, the growth inhibition may for example be a growth inhibition at a concentration of 500 ng/ml.

As further explained in Examples, sections 4c and 4e, below, the inventors have shown that affinity ligands of the present disclosure may bind their targets at a low concentration. Accordingly, in embodiments of the first aspect, the affinity ligand may bind the subset with an EC50 of less than 100 nM, such as less than 50 nM, such as less than 20 nM, such as less than 10 nM. EC50-measurements may for example be performed according to Examples, section 4c and 4e, below.

In embodiments of the first aspect, the affinity ligand may be an antibody or fragment or derivative thereof. Such antibodies may for example be generated according to the Example sections of the present disclosure.

Further examples of affinity ligands according to the first aspect are given below (“Affinity ligands”).

As a first configuration of the first aspect, there is provided an affinity ligand according to the first aspect for use as a medicament.

There are a number of disorders characterized by the overexpression of HER2, and affinity ligands binding the extracellular domain of HER2 may be used as a medicament for treating, or affecting the progression of, such disorders.

Accordingly, as a second configuration of the first aspect, there is provided an affinity ligand according to the first aspect for treatment of a mammalian subject having, or suspected of having, a disorder characterized by the overexpression of HER2. Examples of different disorders characterized by the overexpression of HER2 according to the first aspect are discussed below (“HER2 disorders”).

In the context of the present disclosure, “a mammalian subject having a breast cancer” refers to a mammalian subject having a primary or secondary breast tumor or a mammalian subject which has had a tumor removed from the breast, wherein the removal of the tumor refers to killing or removing the tumor by any type of surgery or therapy. “Breast tumor” includes ductal carcinoma in situ (DCIS). In the method and use aspects of the present disclosure, or in the “product for use”-configurations of the present disclosure, “a mammalian subject having a breast cancer” also includes the case wherein the mammalian subject is suspected of having a breast cancer at the time of the performance of the use or method and the breast cancer diagnosis is established later.

Further, in the context of the present disclosure, “a mammalian subject suspected of having a breast cancer” may for example be a subject presenting typical breast cancer syndrome(s) and/or indicators of high risk for breast cancer, such as an earlier breast cancer or hereditary characteristics, e.g. a history of breast cancer in the family. The risk may also be assessed according to a model, such as the Gail model.

It has been reported that in cancer patients treated with an anti-HER2 antibody, the cancer frequently develops resistance to the anti-HER2 antibody. Consequently, a “new” affinity ligand targeting another part of the extracellular domain of HER2 than the anti-HER2 antibody may be suitable for further treatment of patients having such cancers.

Accordingly, in embodiments of the second configuration of the first aspect, the subject may have been treated by a therapeutic antibody capable of selective interaction with HER2, such as the extracellular domain of HER2, which therapeutic antibody is different from the affinity ligand.

Further, in such embodiments, the disorder characterized by the overexpression of HER2 may for example be a cancer, such as a breast cancer, e.g., a metastatic breast cancer, that has developed resistance to the therapeutic antibody.

For example, the therapeutic antibody capable of selective interaction with HER2 may be trastuzumab or pertuzumab.

As a second aspect of the present invention, there is provided a composition comprising an affinity ligand according to the first aspect and a second affinity ligand capable of selective interaction with a second subset of 73 consecutive amino acid residues or less from extracellular domains 2 and 3 of HER2 (SEQ ID NO:7), said second subset comprising the amino acid sequence of SEQ ID NO:12, SEQ ID NO:13 and/or SEQ ID NO:14.

This second aspect is based on, but not limited to, the inventors' insight that a combination of antibodies targeting two different parts of extracellular domains 2 and 3 of HER2 may result in a greater growth inhibiting effect than antibodies targeting only one of the parts. This is further discussed in Examples, section 5d-f, below.

Examples of different types of second affinity ligands according to the second aspect are given below (“Affinity ligands”).

The sequences SEQ ID NO:21-34 have been found to interact with msAb-N.

Accordingly, in embodiments of the second aspect, the second subset may be selected from the amino acid sequences of the group consisting of SEQ ID NO:21-34.

Further, SEQ ID NO:21-34 are 26, 44, 27, 45, 19, 39, 23, 31, 70, 22, 22, 23, 38 and 23 amino acid residues long, respectively (see also FIG. 7). Also, the identified epitopes SEQ ID NO:12-14 are 8, 10 and 16 amino acid residues long, respectively.

Therefore, in embodiments of the second aspect, the second subset may be 70 amino acid residues or less, such as 55 amino acid residues or less, such as 45 amino acid residues or less, such as 44 amino acid residues or less, such as 39 amino acid residues or less, such as 38 amino acid residues or less, such as 31 amino acid residues or less, such as 27 amino acid residues or less, such as 26 amino acid residues or less, such as 23 amino acid residues or less, such as 22 amino acid residues or less, such as 19 amino acid residues or less, such as 16 amino acid residues or less, such as 10 amino acid residues or less, such as 8 amino acid residues or less.

Also, the second subset of the embodiments of the second aspect may for example be 8 amino acid residues or more, such as 10 amino acid residues or more. This is further discussed above.

In embodiments of the second aspect, the second subset may consist of amino acid residues 39-111 of the sequence SEQ ID NO:4.

As further explained in Examples, section 5d-f, below, the inventors have found that combinations of antibodies of the present disclosure may inhibit growth of breast cancer. Accordingly, in embodiments of the second aspect, the composition may inhibit growth of human breast cancer cells, such as human breast cancer cells in culture. For example, the composition may inhibit growth of human breast cancer cells in culture by 20-100%, such as by 30-100%, relative to an affinity ligand not capable of selective interaction with the extracellular domain of HER2, such as an antibody capable of selective interaction with the intracellular domain of HER2. It is within the capabilities of the skilled artisan to perform measurements yielding such relative growth inhibition value and to adapt such measurements to a specific case. The human breast cancer cells may for example be BT474 breast cancer cells. And further, the growth inhibition may for example be a growth inhibition at a concentration of 500 ng/ml. An example of a growth inhibition measurement is described above in connection with the first aspect.

As further explained in Examples, sections 4c and 4e, below, the inventors have shown that antibodies of the present disclosure may bind their targets at a low concentration. Accordingly, in embodiments of the second aspect, the second affinity ligand may bind the second subset with an EC50 of less than 100 nM, such as less than 50 nM, such as less than 20 nM, such as less than 10 nM. Such EC50-measurements may for example be performed according to Examples, sections 4c and 4e, below.

In embodiments of the second aspect, the second affinity ligand may be an antibody or fragment or derivative thereof. Such an antibody may for example be generated according the Example sections of the present disclosure. Other types of affinity ligands suitable for the second affinity ligand of the second aspect are discussed below (“Affinity ligands”).

In order to strengthen and/or prolong the effect of an affinity ligand according to the first aspect or a composition according to the second aspect or to counteract the development of resistance, the affinity ligand or composition may be combined with a tyrosine kinase inhibitor targeted against HER2. Accordingly, as a variant of the second aspect, there is provided a composition comprising: an affinity ligand according to the first aspect or a composition according to the second aspect; and a tyrosine kinase inhibitor against HER2. The tyrosine kinase inhibitor against HER2 may for example be lapatinib, gefitinib or erlotinib.

As a first configuration of the second aspect, there is provided a composition according to the second aspect for use as a medicament.

There are a number of disorders characterized by the overexpression of HER2, and compositions comprising affinity ligands binding the extracellular domain of HER2 may be used as a medicament for treating, or affecting the progression of, such disorders.

Accordingly, as a second configuration of the second aspect, there is provided a composition according to the second aspect for treatment of a mammalian subject having, or suspected of having, a disorder characterized by the overexpression of HER2. Examples of different disorders characterized by the overexpression of HER2 according to the second aspect are discussed below (“HER2 disorders”).

As mentioned above, it has been reported that in cancer patients treated with an anti-HER2 antibody, the cancer may develop resistance to the anti-HER2 antibody. Consequently, a composition comprising antibodies targeting other parts of the extracellular domain of HER2 than the anti-HER2 antibody may be suitable for further treatment of patients having such cancers.

Accordingly, in embodiments of the second configuration of the second aspect, the subject may have been treated by a therapeutic antibody capable of selective interaction with HER2, such as the extracellular domain of HER2, which therapeutic antibody is different from the affinity ligand or the second affinity ligand.

Further, in such embodiments, the disorder characterized by the overexpression of HER2 may for example be a cancer, such as a breast cancer, e.g., a metastatic breast cancer, that have developed resistance to the therapeutic antibody.

For example, the therapeutic antibody capable of selective interaction with HER2 may be trastuzumab or pertuzumab.

As a third aspect of the present disclosure, there is provided an isolated polypeptide consisting of 37 consecutive amino acid residues or less from extracellular domains 2 and 3 of HER2 (SEQ ID NO:7) and comprising the amino acid sequence LQVF (SEQ ID NO:8) and/or ESFDGD (SEQ ID NO:9).

This third aspect of the present disclosure is based on, but not limited to, the inventors' insight that certain parts of the extracellular domain of HER2 is particularly interesting, e.g. as a therapeutic target, and that fragments comprising or consisting of such parts may be utilized for production, selection or purification of therapeutic means.

A polypeptide fragment of 26 amino acid residues (SEQ ID NO:16), and shorter fragments, such as a fragment of 21 amino acid residues (SEQ ID NO:19), a fragment of 12 amino acid residues (SEQ ID NO:20), two fragments of 9 amino acid residues (SEQ ID NO:15 and 18) and two fragments of 8 amino acid residues (SEQ ID NO:11 and 17), have been found to interact with antibodies exhibiting a growth inhibiting effect.

Therefore, in embodiments of the third aspect, the polypeptide may consist of 31 amino acid residues or less, such as 26 amino acid residues or less, such as 21 amino acid residues or less, such as 16 amino acid residues or less, such as 12 amino acid residues or less, such as 9 amino acid residues or less, such as 8 amino acid residues or less.

A sufficient interaction between an affinity ligand, such as an affinity ligand of the first aspect, and the polypeptide may in some cases require amino acid sequences of various lengths. Accordingly, in embodiments of the third aspect, the polypeptide may consist of 6 amino acid residues or more, such as 8 amino acid residues or more, such as 10 amino acid residues or more.

As shown in Examples, sections 1 and 2, below, the immunization yielding the antibodies having a growth inhibiting effect was performed using an antigen (SEQ ID NO:1) in which the last four amino acid residues at the C-terminal end were LQVF. Accordingly, in embodiments of the third aspect, if the polypeptide comprises the sequence LQVF, it has 2 amino acid residues or less on the C-terminal side of LQVF, such as no amino acid residues on the C-terminal side of LQVF. That is, in some embodiments, the C-terminal of the polypeptide may consist of . . . LQVFET (two amino acid residues on the C-terminal side of LQVF), . . . LQVFE (one amino acid residue on the C-terminal side of LQVF) or . . . LQVF (no amino acid residues on the C-terminal side of LQVF).

A number of fragments (SEQ ID NO:11 and 15-20) have been found to interact with antibodies exhibiting a growth inhibiting effect, i.e. msAb-C (see Examples, section 3). Accordingly, in embodiments of the third aspect, the polypeptide may be selected from the group consisting of the sequences SEQ ID NO:11 and 15-20.

In further embodiments of the third aspect, the polypeptide may comprise the sequence LQVF (SEQ ID NO:8). In such embodiments, the polypeptide may for example be selected from the group consisting of sequences SEQ ID NO:16 and 20. SEQ ID NO:16 and 20 comprise LQVF.

Further, in embodiments of the third aspect, the polypeptide may comprise the sequence ESFDGD (SEQ ID NO:9). In such embodiments, the polypeptide may for example be selected from the group consisting of SEQ ID NO:11 and 15-19.

Still further, in embodiments of the third aspect, the polypeptide may comprise the sequence PESFDGD (SEQ ID NO:10) or LPESFDGD (SEQ ID NO:11).

In embodiments of the third aspect, the polypeptide may consist of the sequence of amino acid residues 1-37 of SEQ ID NO:6. (That is a 37 amino acid residues-long subsequence of SEQ ID NO:6 ending with LQVF.)

As a configuration of the third aspect, there is provided a polypeptide according to the third aspect for use as an antigen, such as an antigen for an immunization, e.g. of a non-human mammal.

As a related configuration thereof, there is provided a polypeptide according to the third aspect for use in the preparation of therapeutic antibodies, e.g. therapeutic antibodies for treatment of disorders characterized by overexpression of HER2. Examples of different disorders characterized by the overexpression of HER2 according to the third aspect are discussed below (“HER2 disorders”).

As a fourth aspect of the present disclosure, there is provided a use of a polypeptide according to the third aspect as an antigen, such as an antigen for an immunization, e.g., an immunization of a non-human mammal.

Uses and methods wherein a polypeptide according to the third aspect is used as an antigen are further discussed below.

As a first configuration of the fourth aspect, there is provided the use of a polypeptide according to the third aspect in the preparation of a therapeutic antibody, such as a therapeutic monoclonal antibody, e.g., a therapeutic chimeric or humanized monoclonal antibody.

As an example, monoclonal antibodies may be made by fusing the spleen cells from a mouse that has been immunized with the polypeptide with myeloma cells. Further, rabbit B-cells may also be used for this purpose.

This mixture of cells may then be diluted, and clones may be grown from single parent cells. The antibodies secreted by the different clones may then be tested for their ability to bind to the polypeptide. Subsequently, a stable and/or productive clone may be grown in culture medium to a high volume.

For example, the DNA that encodes the binding portion of the monoclonal mouse antibodies from the stable clone may be merged with human antibody encoding DNA. Mammalian cell cultures may then be used to express the genetically engineered DNA and produce mouse-human antibodies. Depending on the size of the mouse antibody part, one talks about chimeric antibodies or humanized antibodies. As another example, mice genetically engineered to produce more human-like antibodies may be involved.

Methods for producing monoclonal antibodies and humanization of antibodies are well known to the skilled person.

One reason for the merging with human antibody encoding DNA described above, or involving genetically engineered mice, is to avoid that the human immune system recognizes the antibodies as foreign.

The chimeric antibodies or humanized antibodies may then be used as therapeutic antibodies, e.g. for treatment of disorders characterized by overexpression of HER2.

As a second configuration of the fourth aspect, there is provided the use of a polypeptide according to the third aspect for the selection or purification of an therapeutic affinity ligand for treatment of a disorder characterized by the overexpression of HER2. Examples of different disorders characterized by the overexpression of HER2 according to the fourth aspect are discussed below (“HER2 disorders”).

Examples of therapeutic affinity ligands according to the fourth aspect are given below (“Affinity ligands”).

For example, such use may comprise affinity purification on a solid support onto which the polypeptide has been immobilized. The solid support may for example be arranged in a column. Further, the use may comprise selection of affinity ligands having specificity for a polypeptide according to the third aspect using a solid support onto which the polypeptide has been immobilized. Such solid support may be 96 well plates, magnetic beads, agarose beads or sepharose beads. Further, the use may comprise analysis of affinity ligands on a soluble matrix for example using a dextran matrix or use in a surface plasmon resonance instrument, such as a Biacore™ instrument, were the analysis may for example comprise monitoring the affinity for the immobilized polypeptide and a number of potential affinity ligands.

As a third configuration of the fourth aspect, there is provided the use of a polypeptide according to the third aspect as a therapeutic target.

As a fifth aspect, there are provided uses of the affinity ligand according to the first aspect.

As a first configuration of the fifth aspect, there is provided the use of an affinity ligand according to the first aspect as a medicament.

As a second configuration of the fifth aspect, there is provided the use of an affinity ligand according to first aspect for the manufacture of a medicament for treatment of a mammalian subject having, or suspected of having, a disorder characterized by the overexpression of HER2. Examples of different disorders characterized by the overexpression of HER2 according to the fifth aspect are discussed below (“HER2 disorders”).

In embodiments of the second configuration of the fifth aspect, the subject may have been treated by a therapeutic antibody capable of selective interaction with HER2, such as the extracellular domain of HER2, which therapeutic antibody is different from the affinity ligand.

Also, in embodiments of the second configuration of the fifth aspect, the disorder characterized by the overexpression of HER2 may be a cancer, such as a breast cancer, e.g., a metastatic breast cancer, that has developed resistance to the therapeutic antibody.

Details of the first and second configurations of the fifth aspect is further are as discussed above in connection with the first and second configuration of the first aspect.

As a sixth aspect of the present disclosure, there is provided a method for identification of an affinity ligand for treatment of a disorder characterized by the overexpression of HER2, comprising the steps of:
a) contacting a polypeptide comprising a subset according to the first aspect with a putative affinity ligand; and
b) determining whether the putative affinity ligand binds to the subset.

In embodiments of the sixth aspect, step a) may be:
contacting a polypeptide according to the third aspect with a putative affinity ligand in conditions that enable binding; and step b) may be:
determining whether the putative affinity ligand binds to the polypeptide.

Examples of different disorders characterized by the overexpression of HER2 according to the sixth aspect are given below (“HER2 disorders”).

Further, examples of affinity ligands according to the sixth aspect are given below (“Affinity ligands”).

This sixth aspect is based on, but not limited to, the inventors' insight that protein fragments corresponding to the identified target sequences of the extracellular domain of HER2 may be useful for identification or selection of therapeutic affinity ligands.

In embodiments of the sixth aspect wherein the disorder is a cancer, such as a breast cancer, the method may further comprise the step:
c) determining whether the putative affinity ligand inhibits growth or induces apoptosis of cancer cells, such as breast cancer cells, e.g., BT474 breast cancer cells.

For example, the criterion of step c) may be that the putative affinity ligand inhibits growth by 20% or more, such as 30% or more, as compared to an antibody targeting the intracellular part of HER2. For example, the putative affinity ligand may inhibit growth at a concentration of 250 or 500 ng/ml.

Such determination may for example be performed as in Examples, section 5, below.

As a first configuration of the sixth aspect, there is provided a method for identification of one or more affinity ligands for treatment of a disorder characterized by the overexpression of HER2, comprising the steps of:
a) contacting a polypeptide according to the third aspect with one or more putative affinity ligands; and
b) identifying affinity ligands that bind to the polypeptide.

As a second configuration of the sixth aspect, there is provided a method for producing a clone, e.g., a clone expressing a therapeutic antibody for treatment of a disorder characterized by the overexpression of HER2, comprising:
a) providing cells obtained from a mammal which has been immunized with an antigen comprising a subset according to the first aspect, which cells comprise DNA encoding an antibody capable of selective interaction with the subset; and
b) fusing said cells with myeloma cells to obtain at least one clone.

In the context of the present disclosure, “a clone” refers to a group of identical cells that share a common ancestry, i.e. are derived from the same mother cell.

For example, step b) may comprise culturing.

In embodiments of the second configuration of the sixth aspect, the method further comprises the step:
a′) immunizing the mammal with the antigen,
   wherein step a′) precedes step a).

For example, the mammal of step a) may be a non-human mammal.

Further, the cells provided in step a) may for example be spleen cells. Also, the mammal of step a) may for example be a mouse. Consequently, the cells provided in step a) may for example be spleen cells from a mouse.

Alternatively, the cells provided in step a) may for example be B-cells. Further, the mammal of step a) may for example be a rabbit. Consequently, the cells provided in step a) may for example be rabbit B-cells.

In embodiments of the second configuration of the sixth aspect, the method may further comprise the step:
c) selecting a clone from step b) which secretes antibodies capable of selective interaction with the subset.

Further, in embodiments of the second configuration of the sixth aspect, the antigen may consist of a polypeptide according to the third aspect. In such embodiments, a clone which secretes antibodies capable of selective interaction with the antigen is selected, if the method comprises step c).

Also, in embodiments of the second configuration of the sixth aspect, the method may further comprise the step:
d) providing a clone obtained in step b) or selected in step c), and merging DNA from the clone, which DNA encodes at least the part of an antibody expressed by the clone that selectively interacts with the subset, with human antibody encoding DNA; and
e) incorporating the merged DNA from step d) in cells to obtain a clone for expression of a therapeutic antibody for treatment of a disorder characterized by the overexpression of HER2.

For example, step e) may comprise culturing.

The clone of step e) may for example be a mammalian cell line. The therapeutic antibodies expressed by the clone of step e) may for example be chimeric or humanized antibodies.

As a third configuration of the sixth aspect, there is provided a method of producing an affinity ligand, such as an antibody, e.g. a therapeutic antibody, comprising: identifying an affinity ligand using the method according to the sixth aspect; and producing said identified affinity ligand. It is within the capabilities of the skilled person, especially if guided by the teachings of the present disclosure, to produce such identified affinity ligand.

As a fourth configuration of the sixth aspect, there is provided a method of producing an affinity ligand, such as an antibody, e.g. a therapeutic antibody, comprising: producing a clone using the method according to the second configuration of the sixth aspect; and obtaining said affinity ligand from said clone. It is within the capabilities of the skilled person, especially if guided by the teachings of the present disclosure, to obtain the affinity ligand from the clone. For example, obtaining said affinity ligand from the clone may comprise initiating expression of the affinity ligand, e.g. an antibody, and harvesting of the subsequently secreted affinity ligand (e.g. antibody).

As a seventh aspect of the present disclosure, there is provided a method of treatment of a mammalian subject having, or suspected of having, a disorder characterized by the overexpression of HER2, comprising administering an effective amount of an affinity ligand according to the first aspect or a composition according to the second aspect to the subject.

Examples of different disorders characterized by the overexpression of HER2 according to the seventh aspect are discussed below (“HER2 disorders”).

In embodiments of the seventh aspect, the method may further comprise administering a tyrosine kinase inhibitor against HER2 to the subject.

Further, in embodiments of the seventh aspect, the treatment may be a pre-surgical treatment. Consequently, e.g., a subject suspected of having a breast cancer or having a high risk of breast cancer recurrence or a subject having a breast cancer surgery scheduled may be treated according to the seventh aspect.

Alternatively, in embodiments of the seventh aspect, the treatment may be a post-surgical treatment.

Further, the treatment may be pre- and post-surgical treatment, e.g., a first effective amount of the affinity ligand or composition may be administered to the subject before surgical removal of a breast cancer tumor and a second effective amount of the affinity ligand or composition may be administered to the subject after the surgical removal of the breast cancer tumor.

In embodiments of the seventh aspect, the subject may have been treated by a therapeutic antibody capable of selective interaction with HER2, such as the extracellular domain of HER2, which therapeutic antibody is different from the affinity ligand. Such therapeutic antibody may for example be trastuzumab or pertuzumab.

In such embodiments of the seventh aspect, the disorder characterized by the overexpression of HER2 may be a cancer, such as a breast cancer, that has developed resistance to the therapeutic antibody.

As an eighth aspect of the present invention, there is provided an article of manufacture, comprising: a container; a composition within the container comprising an affinity ligand according to the first aspect or a composition according to the second aspect; and a label on or associated with the container that indicates that said composition can be used for treating a disorder characterized by the overexpression of HER2.

For example, the container may be a bottle, vial or syringe. The container may be formed from a variety of materials such as glass or plastic. The container holds an affinity ligand or composition which is effective for treating the disorder and may have a sterile access port. For example the container may be an intravenous solution bag or a vial having a stopper pierceable by a hypodermic injection needle. For example, the article of manufacture may further comprise a second container comprising a pharmaceutically acceptable buffer, such as phosphate-buffered saline, Ringer's solution or dextrose solution. Also, the article of manufacture may further include other materials desirable from a commercial and user standpoint, including other buffers, diluents, filters, needles, and syringes. For example, the article of manufacture may, in addition, comprise a package insert with instructions for use. This may for example be instructions for pre- and/or post-surgical use and/or instructions for administration to a subject having a cancer which has developed resistance to a therapeutic anti-HER2 antibody.

Examples of different disorders characterized by the overexpression of HER2 according to the eighth aspect are discussed below (“HER2 disorders”).

As a ninth aspect of the present disclosure, there is provided an affinity ligand, which is the second affinity ligand of the second aspect.

As a first configuration of the ninth aspect, there is provided an affinity ligand according to the ninth aspect for use as a medicament.

There are a number of disorders characterized by the overexpression of HER2, and affinity ligands binding the extracellular domain of HER2 may be used as a medicament for treating, or affecting the progression of, such disorders.

Accordingly, as a second configuration of the ninth aspect, there is provided the affinity ligand according to the ninth aspect for treatment of a disorder characterized of overexpression of HER2. Examples of different disorders characterized by the overexpression of HER2 according to the ninth aspect are discussed below (“HER2 disorders”).

As mentioned above, it has been reported that in cancer patients treated with an anti-HER2 antibody, the cancer frequently develops resistance to the anti-HER2 antibody. Consequently, a “new” affinity ligand targeting another part of the extracellular domain of HER2 than the anti-HER2 antibody may be suitable for further treatment of patients having such cancers.

Accordingly, in embodiments of the second configuration of the ninth aspect, the subject may have been treated by a therapeutic antibody capable of selective interaction with HER2, such as the extracellular domain of HER2, which therapeutic antibody is different from the affinity ligand according to the ninth aspect.

Further, in such embodiments, the disorder characterized by the overexpression of HER2 may for example be a cancer, such as a breast cancer, e.g., a metastatic breast cancer, that have developed resistance to the therapeutic antibody.

For example, the therapeutic antibody capable of selective interaction with HER2 may be trastuzumab or pertuzumab.

As a tenth aspect of the present disclosure, there is provided an isolated polypeptide consisting of 73 consecutive amino acid residues or less from extracellular domains 2 and 3 of HER2 (SEQ ID NO:7) and comprising the amino acid sequence of SEQ ID NO:12, SEQ ID NO:13 and/or SEQ ID NO:14.

This tenth aspect of the present disclosure is based on, but not limited to, the inventors' insight that certain parts of the extracellular domain of HER2 is particularly interesting, e.g. as a therapeutic target, and that fragments comprising or consisting of such parts may be utilized for production, selection or purification of therapeutic means.

In embodiments of the tenth aspect, the polypeptide may consist of a amino acid sequence selected from the group consisting of SEQ ID NO:21-34.

The polypeptides SEQ ID NO:21-34, which have been found to interact with msAb-N, are 26, 44, 27, 45, 19, 39, 23, 31, 70, 22, 22, 23, 38 and 23 amino acid residues long, respectively (see also FIG. 7). Also, the identified epitopes SEQ ID NO:12-14 are 8, 10 and 16 amino acid residues long, respectively.

Therefore, in embodiments of the tenth aspect, the polypeptide may consist of 70 amino acid residues or less, such as 55 amino acid residues or less, such as 45 amino acid residues or less, such as 44 amino acid residues or less, such as 39 amino acid residues or less, such as 38 amino acid residues or less, such as 31 amino acid residues or less, such as 27 amino acid residues or less, such as 26 amino acid residues or less, such as 23 amino acid residues or less, such as 22 amino acid residues or less, such as 19 amino acid residues or less, such as 16 amino acid residues or less, such as 10 amino acid residues or less, such as 8 amino acid residues or less.

As mentioned above, a sufficient interaction between an affinity ligand, such as an affinity ligand of the ninth aspect, and the polypeptide may in some cases require amino acid sequences of various lengths. Accordingly, in embodiments of the tenth aspect, the polypeptide may consist of 6 amino acid residues or more, such as 8 amino acid residues or more, such as 10 amino acid residues or more.

In further embodiments of the tenth aspect, the polypeptide may comprise the sequence SEQ ID NO:12. In such embodiments, the polypeptide may for example consist of any one of the sequences SEQ ID NO:21-34 that comprises SEQ ID NO:12.

In further embodiments of the tenth aspect, the polypeptide may comprise the sequence SEQ ID NO:13. In such embodiments, the polypeptide may for example consist of any one of the sequences SEQ ID NO:21-34 that comprises SEQ ID NO:13.

In further embodiments of the tenth aspect, the polypeptide may comprise the sequence SEQ ID NO:14. In such embodiments, the polypeptide may for example consist of any one of the sequences SEQ ID NO:21-34 that comprises SEQ ID NO:14.

In embodiments of the tenth aspect, the polypeptide may consist of amino acid residues 39-111 of the sequence SEQ ID NO:4.

As a configuration of the tenth aspect, there is provided a polypeptide according to the tenth aspect for use as an antigen, such as an antigen for an immunization, e.g. of a non-human mammal.

As a related configuration thereof, there is provided a polypeptide according to the tenth aspect for use in the preparation of therapeutic antibodies, e.g. therapeutic antibodies for treatment of disorders characterized by overexpression of HER2. Examples of different disorders characterized by the overexpression of HER2 according to the tenth aspect are discussed below (“HER2 disorders”).

As a eleventh aspect of the present disclosure, there is provided a use of a polypeptide according to the tenth aspect as an antigen, such as an antigen for an immunization, e.g., an immunization of a non-human mammal.

Uses and methods wherein a polypeptide according to the tenth aspect is used as an antigen are further discussed below.

As a first configuration of the eleventh aspect, there is provided the use of a polypeptide according to the tenth aspect in the preparation of a therapeutic antibody, such as therapeutic monoclonal antibody, e.g., a therapeutic chimeric or humanized monoclonal antibody. This is further discussed above in connection with the first configuration of the fourth aspect.

As a second configuration of the eleventh aspect, there is provided the use of a polypeptide according to the tenth aspect for the selection or purification of a therapeutic affinity ligand for treatment of a disorder characterized by the overexpression of HER2. Examples of different disorders characterized by the overexpression of HER2 according to the eleventh aspect are discussed below (“HER2 disorders”).

For example, such use may comprise affinity purification on a solid support onto which the polypeptide has been immobilized. The solid support may for example be arranged in a column. Further, the use may comprise selection of affinity ligands having specificity for a polypeptide according to the tenth aspect using a solid support onto which the polypeptide has been immobilized. Such solid support may be 96 well plates, magnetic beads, agarose beads or sepharose beads. Further, the use may comprise analysis of affinity ligands on a soluble matrix for example using a dextran matrix or use in a surface plasmon resonance instrument, such as a Biacore™ instrument, were the analysis may for example comprise monitoring the affinity for the immobilized polypeptide and a number of potential affinity ligands.

Examples of therapeutic affinity ligands according to the eleventh aspect are given below (“Affinity ligands”).

As a third configuration of the eleventh aspect, there is provided the use of a polypeptide according to the tenth aspect as a therapeutic target.

As a twelfth aspect, there are provided uses of the affinity ligand according to the ninth aspect.

As a first configuration of the twelfth aspect, there is provided the use an affinity ligand according to the ninth aspect as a medicament.

As a second configuration of the twelfth aspect, there is provided the use of an affinity ligand according to ninth aspect for the manufacture of a medicament for treatment of a mammalian subject having, or suspected of having, a disorder characterized by the overexpression of HER2. Examples of different disorders characterized by the overexpression of HER2 according to the twelfth aspect are discussed below (“HER2 disorders”).

In embodiments of the second configuration of the twelfth aspect, the subject may have been treated by an therapeutic antibody capable of selective interaction with HER2, such as the extracellular domain of HER2, which therapeutic antibody is different from the affinity ligand.

Also, in embodiments of the second configuration of the twelfth aspect, the disorder characterized by the overexpression of HER2 may be a cancer, such as a breast cancer, e.g., a metastatic breast cancer, that has developed resistance to the therapeutic antibody.

The subject-matter of the first and second configuration of the twelfth aspect is further discussed above in connection with the first and second configuration of the ninth aspect.

As a thirteenth aspect of the present disclosure, there is provided a method for identification of an affinity ligand for treatment of a disorder characterized by the overexpression of HER2, comprising the steps of:
a) contacting a Polypeptide comprising a second subset according to the second aspect with a putative affinity ligand; and
b) determining whether the putative affinity ligand binds to the second subset.

In embodiments of the thirteenth aspect, step a) may be:
contacting a polypeptide according the tenth aspect with a putative affinity ligand in conditions that enable binding;

and step b) may be:
determining whether the putative affinity ligand binds to the polypeptide.

Examples of different disorders characterized by the overexpression of HER2 according to the thirteenth aspect are given below (“HER2 disorders”).

Further, examples of affinity ligands according to the thirteenth aspect are given below (“Affinity ligands”).

This thirteenth aspect is based on, but not limited to, the inventors' insight that protein fragments corresponding to the identified target sequences of the extracellular domain of HER2 may be useful for identification or selection of therapeutic affinity ligands.

In embodiments of the thirteenth aspect wherein the disorder is a cancer, such as a breast cancer, the method may further comprise the step:
c) determining whether the putative affinity ligand inhibits growth or induces apoptosis of cancer cells, such as breast cancer cells, e.g., BT474 breast cancer cells.

For example, the criterion of step c) may be that the putative affinity ligand inhibits growth more than an antibody targeting the intracellular part of HER2. For example, the putative affinity ligand may inhibit growth at a concentration of 250 or 500 ng/ml.

Such determination may for example be performed as in Examples, section 5, below.

As a first configuration of the thirteenth aspect, there is provided a method for identification of one or more affinity ligands for treatment of a disorder characterized by the overexpression of HER2, comprising the steps of:
a) contacting a polypeptide according to the tenth aspect with one or more putative affinity ligands; and
b) identifying affinity ligands that bind to the polypeptide.

As a second configuration of the thirteenth aspect, there is provided a method for producing a clone, e.g., a clone expressing a therapeutic antibody for treatment of a disorder characterized by the overexpression of HER2, comprising:
a) providing cells obtained from a mammal which has been immunized with an antigen comprising the second subset according to the second aspect, which cells comprise DNA encoding an antibody capable of selective interaction with the subset; and
b) fusing said cells with myeloma cells to obtain at least one clone.

For example, step b) may comprise culturing.

In embodiments of the second configuration of the thirteenth aspect, the method further comprises the step:
a′) immunizing the mammal with the antigen,
   wherein step a′) precedes step a).

For example, the mammal of step a) may be a non-human mammal.

Further, the cells provided in step a) may for example be spleen cells. Also, the mammal of step a) may for example be a mouse. Consequently, the cells provided in step a) may for example be spleen cells from a mouse.

Alternatively, the cells provided in step a) may for example be B-cells. Further, the mammal of step a) may for example be a rabbit. Consequently, the cells provided in step a) may for example by rabbit B-cells.

In embodiments of the second configuration of the thirteenth aspect, the method may further comprise the step:
c) selecting a clone from step b) which secretes antibodies capable of selective interaction with the subset.

Further, in embodiments of the second configuration of the thirteenth aspect, the antigen may consist of a polypeptide according to the tenth aspect. In such embodiments, a clone which secretes antibodies capable of selective interaction with the antigen is selected, if the method comprises step c).

Also, in embodiments of the second configuration of the thirteenth aspect, the method may further comprise the step:
d) providing a clone obtained in step b) or selected in step c), and merging DNA from the clone, which DNA encodes at least the part of an antibody expressed by the clone that selectively interacts with the subset, with human antibody encoding DNA; and
f) incorporating the merged DNA from step d) in cells to obtain a clone for expression of a therapeutic antibody for treatment of a disorder characterized by the overexpression of HER2.

For example, step e) may comprise culturing.

The clone of step e) may for example be a mammalian cell line. The therapeutic antibodies expressed by the clone of step e) may for example be chimeric or humanized antibodies.

As a third configuration of the thirteenth aspect, there is provided a method of producing an affinity ligand, such as an antibody, e.g. a therapeutic antibody, comprising: identifying an affinity ligand using the method according to the thirteenth aspect; and producing said identified affinity ligand. It is within the capabilities of the skilled person, especially if guided by the teachings of the present disclosure, to produce such identified affinity ligand.

As a fourth configuration of the sixth aspect, there is provided a method of producing an affinity ligand, such as an antibody, e.g. a therapeutic antibody, comprising: producing a clone using the method according to the second configuration of the thirteenth aspect; and obtaining said affinity ligand from said clone. It is within the capabilities of the skilled person, especially if guided by the teachings of the present disclosure, to obtain the affinity ligand from the clone. For example, obtaining said affinity ligand from the clone may comprise initiating expression of the affinity ligand, e.g. an antibody, and harvesting of the subsequently secreted affinity ligand (e.g. antibody).

As a fourteenth aspect of the present disclosure, there is provided a method of treatment of a mammalian subject having, or suspected of having, a disorder characterized by the overexpression of HER2, comprising administering an effective amount of an affinity ligand according to the ninth aspect to the subject.

Examples of different disorders characterized by the overexpression of HER2 according to the fourteenth aspect are discussed below (“HER2 disorders”).

In embodiments of the fourteenth aspect, the method may further comprise administering a tyrosine kinase inhibitor against HER2 to the subject.

Further, in embodiments of the fourteenth aspect, the treatment may be a pre-surgical treatment. Consequently, e.g., a subject suspected of having a breast cancer or having a high risk of breast cancer recurrence or a subject having a breast cancer surgery scheduled may be treated according to the fourteenth aspect.

Alternatively, in embodiments of the fourteenth aspect, the treatment may be a post-surgical treatment.

Further, the treatment may be pre- and post-surgical treatment, e.g., a first effective amount of the affinity ligand may be administered to the subject before surgical removal of a breast cancer tumor and a second effective amount of the affinity ligand may be administered to the subject after the surgical removal of the breast cancer tumor.

In embodiments of the fourteenth aspect, the subject may have been treated by a therapeutic antibody capable of selective interaction with HER2, such as the extracellular domain of HER2, which therapeutic antibody is different from the affinity ligand. Such therapeutic antibody may for example be trastuzumab or pertuzumab.

In such embodiments of the fourteenth aspect, the disorder characterized by the overexpression of HER2 may be a cancer, such as a breast cancer, that has developed resistance to the therapeutic antibody.

As a fifteenth aspect of the present invention, there is provided an article of manufacture, comprising: a container; a composition within the container comprising an affinity ligand according to the ninth aspect; and a label on or associated with the container that indicates that said composition can be used for treating a disorder characterized by the overexpression of HER2.

Examples of different disorders characterized by the overexpression of HER2 according to the fifteenth aspect are discussed below (“HER2 disorders”).

Affinity Ligands

The affinity ligands according to the various embodiments of the above aspects of the present disclosure, such as the affinity ligand of the first aspect and the second affinity ligand of the second aspect, may independently be any type of affinity ligands.

Nevertheless, examples of such affinity ligands that may prove useful in the context of the present disclosure are given below.

Thus, in some embodiments of the above aspects, the affinity ligands may be independently selected from the group consisting of antibodies, fragments thereof and derivatives thereof, i.e., affinity ligands based on an immunoglobulin scaffold. For example, the antibodies may be isolated and/or mono-specific. Antibodies comprise monoclonal and polyclonal antibodies of any origin, including murine, rabbit, human and other antibodies, as well as chimeric antibodies comprising sequences from different species, such as partly humanized antibodies or humanized antibodies, such as partly humanized or humanized mouse antibodies. Initially, antibodies are produced by immunization of animals with the antigen of choice; polyclonal antibodies are then purified from blood/sera, whereas monoclonal antibodies of defined specificity can be produced using the hybridoma technology developed by Köhler and Milstein (Köhler G and Milstein C (1976) Eur. J. Immunol. 6:511-519). Antibody fragments and derivatives comprise Fab fragments, consisting of the first constant domain of the heavy chain (CH1), the constant domain of the light chain (CL), the variable domain of the heavy chain (VH) and the variable domain of the light chain (VL) of an intact immunoglobulin protein; Fv fragments, consisting of the two variable antibody domains VH and VL (Skerra A and Plückthun A (1988) Science 240:1038-1041); single chain Fv fragments (scFv), consisting of the two VH and VL domains linked together by a flexible peptide linker (Bird R E and Walker B W (1991) Trends Biotechnol. 9:132-137); Bence Jones dimers (Stevens F J et al. (1991) Biochemistry 30:6803-6805); camelid heavy-chain dimers (Hamers-Casterman C et al. (1993) Nature 363:446-448) and single variable domains (Cai X and Garen A (1996) Proc. Natl. Acad. Sci. U.S.A. 93:6280-6285; Masat L et al. (1994) Proc. Natl. Acad. Sci. U.S.A. 91:893-896), and single domain scaffolds like e.g., the New Antigen Receptor (NAR) from the nurse shark (Dooley H et al. (2003) Mol. Immunol. 40:25-33) and minibodies based on a variable heavy domain (Skerra A and Plückthun A (1988) Science 240:1038-1041).

Antibodies, as well as their fragments and derivatives, represent the traditional choice of affinity ligands in therapeutic applications. However, those of skill in the art know that, e.g., due to the increasing demand of high throughput generation of selective binding ligands and low cost production systems, new biomolecular diversity technologies have been developed during the last decade. This has enabled a generation of novel types of affinity ligands of both immunoglobulin and non-immunoglobulin origin that may be useful as binding ligands in e.g. therapeutic applications and can be used instead of, or together with, immunoglobulins.

The biomolecular diversity needed for selection of affinity ligands may be generated by combinatorial engineering of one of a plurality of possible scaffold molecules, and specific and/or selective affinity ligands are then selected using a suitable selection platform. The scaffold molecule may be of immunoglobulin protein origin (Bradbury A R and Marks J D (2004) J. Immunol. Meths. 290:29-49), of non-immunoglobulin protein origin (Nygren P Å and Skerra A (2004) J. Immunol. Meths. 290:3-28), or of an oligonucleotide origin (Gold L et al. (1995) Annu. Rev. Biochem. 64:763-797).

A large number of non-immunoglobulin protein scaffolds have been used as supporting structures in development of novel binding proteins. Non-limiting examples of such structures, useful for generating affinity ligands against the relevant HER2 subsets are staphylococcal protein A and domains thereof and derivatives of these domains, such as protein Z (Nord K et al. (1997) Nat. Biotechnol. 15:772-777); lipocalins (Beste G et al. (1999) Proc. Natl. Acad. Sci. U.S.A. 96:1898-1903); ankyrin repeat domains (Binz H K et al. (2003) J. Mol. Biol. 332:489-503); cellulose binding domains (CBD) (Smith G P et al. (1998) J. Mol. Biol. 277:317-332; Lehtiö J et al. (2000) Proteins 41:316-322); γ crystallines (Fiedler U and Rudolph R, WO01/04144); green fluorescent protein (GFP) (Peelle B et al. (2001) Chem. Biol. 8:521-534); human cytotoxic T lymphocyte-associated antigen 4 (CTLA-4) (Hufton S E et al. (2000) FEBS Lett. 475:225-231; Irving R A et al. (2001) J. Immunol. Meth. 248:31-45); protease inhibitors, such as Knottin proteins (Wentzel A et al. (2001) J. Bacteriol. 183:7273-7284; Baggio R et al. (2002) J. Mol. Recognit. 15:126-134) and Kunitz domains (Roberts B L et al. (1992) Gene 121:9-15; Dennis M S and Lazarus R A (1994) J. Biol. Chem. 269:22137-22144); PDZ domains (Schneider S et al. (1999) Nat. Biotechnol. 17:170-175); peptide aptamers, such as thioredoxin (Lu Z et al. (1995) Biotechnology 13:366-372; Klevenz B et al. (2002) Cell. Mol. Life. Sci. 59:1993-1998); staphylococcal nuclease (Norman T C et al. (1999) Science 285:591-595); tendamistats (McConell S J and Hoess R H (1995) J. Mol. Biol. 250:460-479; Li R et al. (2003) Protein Eng. 16:65-72); trinectins based on the fibronectin type III domain (Koide A et al. (1998) J. Mol. Biol. 284:1141-1151; Xu L et al. (2002) Chem. Biol. 9:933-942); and zinc fingers (Bianchi E et al. (1995) J. Mol. Biol. 247:154-160; Klug A (1999) J. Mol. Biol. 293:215-218; Segal D J et al. (2003) Biochemistry 42:2137-2148).

The above-mentioned examples of non-immunoglobulin protein scaffolds include scaffold proteins presenting a single randomized loop used for the generation of novel binding specificities, protein scaffolds with a rigid secondary structure where side chains protruding from the protein surface are randomized for the generation of novel binding specificities, and scaffolds exhibiting a non-contiguous hyper-variable loop region used for the generation of novel binding specificities.

In addition to non-immunoglobulin proteins, oligonucleotides may also be used as affinity ligands. Single stranded nucleic acids, called aptamers or decoys, fold into well-defined three-dimensional structures and bind to their target with high affinity and specificity (Ellington A D and Szostak J W (1990) Nature 346:818-822; Brody E N and Gold L (2000) J. Biotechnol. 74:5-13; Mayer G and Jenne A (2004) BioDrugs 18:351-359). The oligonucleotide ligands can be either RNA or DNA and can bind to a wide range of target molecule classes.

For selection of the desired affinity ligand from a pool of variants of any of the scaffold structures mentioned above, a number of selection platforms are available for the isolation of a specific novel ligand against a target protein of choice. Selection platforms include, but are not limited to, phage display (Smith GP (1985) Science 228:1315-1317), ribosome display (Hanes J and Plückthun A (1997) Proc. Natl. Acad. Sci. U.S.A. 94:4937-4942), yeast two-hybrid system (Fields S and Song 0 (1989) Nature 340:245-246), yeast display (Gai S A and Wittrup K D (2007) Curr Opin Struct Biol 17:467-473), mRNA display (Roberts R W and Szostak J W (1997) Proc. Natl. Acad. Sci. U.S.A. 94:12297-12302), bacterial display (Daugherty P S (2007) Curr Opin Struct Biol 17:474-480, Kronqvist N et al. (2008) Protein Eng Des Sel 1-9, Harvey B R et al. (2004) PNAS 101(25):913-9198), microbead display (Nord O et al. (2003) J Biotechnol 106:1-13, WO01/05808), SELEX (System Evolution of Ligands by Exponential Enrichment) (Tuerk C and Gold L (1990) Science 249:505-510) and protein fragment complementation assays (PCA) (Remy I and Michnick S W (1999) Proc. Natl. Acad. Sci. U.S.A. 96:5394-5399).

Thus, in embodiments of the above aspects, the affinity ligands may each independently be a non-immunoglobulin affinity ligand derived from any of the protein scaffolds listed above, or an oligonucleotide molecule.

HER2 Disorders

In embodiments of the above aspects (aspects one to fifteen), the disorder characterized by the overexpression of HER2 may be a cancer.

Further, in embodiments of the above aspects, the cancer may be selected from the group consisting of breast cancer, squamos cell cancinoma, lung cancer, such as small cell or non-small cell lung cancer, pancreatic cancer, glioblastoma, cervical cancer, ovarian cancer, vulval cancer, liver cancer, hepatoma, colorectal cancer, such as colon cancer, endometrial carcinoma, salivary gland carcinoma, kidney cancer, thyroid cancer, Wilm's tumor, bladder cancer, endometrial cancer, renal cancer, head and neck cancer, gastric cancer, esophageal cancer and prostate cancer.

For example, the cancer may be selected from the group consisting of breast cancer, lung cancer, pancreatic cancer, colorectal cancer and Wilm's tumor.

The HER2 protein has been reported to be overexpressed in about 20% of all, and in up to 70% of lowly differentiated, breast cancers. Also, the efficiency of anti-HER2 treatment of breast cancer subjects, e.g. subjects having metastatic breast cancer, have been well studied.

Accordingly, in embodiments of the above aspects, the disorder characterized by the overexpression of HER2 may be a breast cancer. For example, the breast cancer may be a metastatic breast cancer.

### BRIEF DESCRIPTION OF THE FIGURES

FIG. 1 shows affinity purification and specificity analyses.

1a shows a schematic drawing of the setup for serial selective affinity purification. Polyclonal antibodies raised against the full-length antigen, here denoted 866 (SEQ ID NO:1) were split into four specific populations: an anti-C-terminal fraction (Ab-C); an anti-M (middle) fraction (Ab-M); anti-N-terminal fraction (Ab-N); and finally a full-length antigen column were connected to collect possible antibodies binding structural epitopes (Ab-S).

1b shows an analysis of binding specificity for the polyclonal antibodies raised against the full-length antigen, here denoted Ab-866 and the antibodies obtained from the split into four specific populations: Ab-C, Ab-M, Ab-N and Ab-S using a Luminex bead array system. A high specificity was revealed for all purified fractions. Black=binding, white=no binding.

1c shows a Luminex bead array competition assay used for estimation of relative affinities of the purified mono-specific antibodies. The interaction between mono-specific antibody and the full antigen (i.e., 866 (SEQ ID NO:1)) immobilized on the bead surface was challenged using an increasing concentration of soluble full-length antigen (i.e., 866 (SEQ ID NO:1)) as competitor protein fragment.

FIG. 2 shows the result of Fluorescence Activated Cell Sorting (FACS) of BT474 cells. FACS of unlabeled cells (2a) and cells labeled with Ab-Intra, a polyclonal antibody (HPA001383) targeting the intracellular part of HER2 and used as a negative control (2b), Ab-866 (2c), Ab-N (2d), Ab-M (2e), Ab-C (2f), trastuzumab (2g) and Ab-S (2h), respectively, were evaluated. Enriched populations with higher fluorescence over unlabeled cells were observed for trastuzumab, msAb-866, msAb-N and msAb-C indicating cell binding, whereas no significant enrichment in fluorescence was seen for msAb-intra or msAb-M.

FIG. 3 shows a dose-response study where BT474 cells were treated with an increasing amount of msAb-866.

FIG. 4 shows a growth inhibition study of BT474 cells using 500 ng/ml of msAb-Intra, msAb-M, msAb-N, msAb-C, msAb-CNM, msAb-866 and msAb-NC, respectively. The presented “Effect” values are relative to cultures treated with msAb-Intra. msAb-N, msAb-C, msAb-CNM, msAb-866 and msAb-NC showed between 14% and 39% cell growth inhibition effect.

FIG. 5 shows a growth inhibition study of BT474 cells using 500 ng/ml of msAb-M, msAb-Intra, msAb-N, trastuzumab, msAb-C and msAb-866, respectively. The presented “Effect” values are relative to cultures treated with msAb-Intra.

FIG. 6 shows an amino acid alignment of a selection of HER2 fragments (SEQ ID NO:11 and 15-20) each comprising one or more of the C-epitopes (SEQ ID NO:8-11) identified through epitope mapping. Some of the sequences start at the upper half of the figure and continue at the lower half.

FIG. 7 shows an amino acid alignment of a selection of HER2 fragments (SEQ ID NO:21-34) each comprising one or more of the N-epitopes (SEQ ID NO:12-14) identified through epitope mapping. Some of the sequences start at the upper half of the figure and continue at the lower half.

### GENERATION OF MONO-SPECIFIC ANTIBODIES AGAINST HER2 AND STUDIES OF THEIR INTERACTION WITH VARIOUS HER FRAGMENTS AND IMPACT ON CANCEROUS CELLS

### 1. Generation of Antigen

a) Materials and Methods

A suitable fragment of the target protein encoded by the EnsEMBL Gene ID ENSG00000141736 was selected using bioinformatic tools with the human genome sequence as template (Lindskog M et al (2005) Biotechniques 38:723-727, EnsEMBL, www.ensembl.org). The fragment was used as template for the production of a 127 amino acid long fragment corresponding to amino acid residues 274-400 (SEQ ID NO:1) of the HER2 protein (SEQ ID NO:2; EnsEMBL entry no. ENSP00000269571). The protein fragment was designed to consist of a unique sequence with low sequence similarity to other human proteins, to minimize unwanted cross reactivity of generated affinity reagents, and still be of a suitable size to allow formation of conformational epitopes and allow efficient expression in bacterial systems.

A fragment of the HER2 gene transcript containing nucleotides 1058-1438 of EnsEMBL entry number ENST00000269571 (SEQ ID NO:3), was isolated using Superscript™ One-Step RT-PCR amplification kit with Platinum® Taq (Invitrogen) and a human total RNA pool panel as template (Human Total RNA Panel IV, BD Biosciences Clontech). Flanking restriction sites NotI and AscI were introduced into the fragment through the PCR amplification primers to allow in-frame cloning into the expression vector (forward primer: TACAACACAGACACGTTTGAG, biotinylated reverse primer: AAACACTTGGAGCTGCTCTG). Resulting biotinylated PCR product was immobilized onto Dynabeads M280 Streptavidin (Dynal Biotech) (Larsson M et al (2000) J. Biotechnol. 80:143-157) and subjected to Not-Asci digestion (New England Biolabs) on solid support by NotI-AscI digestion, ligated into the pAff8c vector (Larsson M et al, supra) in frame with a N-terminal dual affinity tag consisting of a hexahistidyl tag for immobilized metal ion chromatography (IMAC) purification and an immunopotentiating albumin binding protein (ABP) from streptococcal protein G (Sjölander A et al (1997) J. Immunol. Methods 201:115-123; Ståhl Set al (1999) Encyclopedia of Bioprocess Technology Fermentation, Biocatalysis and Bioseparation (Fleckinger M C and Drew S W, eds) John Wiley and Sons Inc., New York, pp 49-63), and transformed into E. coli BL21(DE3) cells (Novagen). The sequences of the clones were verified by dye-terminator cycle sequencing of plasmid DNA amplified using TempliPhi DNA sequencing amplification kit (GE Healthcare, Uppsala, Sweden) according to the manufacturer's recommendations.

BL21(DE3) cells harboring the expression vector were inoculated in 100 ml 30 g/l tryptic soy broth (Merck KGaA) supplemented with 5 g/l yeast extract (Merck KGaA) and 50 mg/l kanamycin (Sigma-Aldrich) by addition of 1 ml of an overnight culture in the same culture medium. The cell culture was incubated in a 1 liter shake flask at 37° C. and 150 rpm until the optical density at 600 nm reached 0.5-1.5. Protein expression was then induced by addition of isopropyl-β-D-thiogalactopyranoside (Apollo Scientific) to a final concentration of 1 mM, and the incubation was continued overnight at 25° C. and 150 rpm. The cells were harvested by centrifugation at 2400 g, and the pellet was re-suspended in 5 ml lysis buffer (7 M guanidine hydrochloride, 47 mM Na2HPO4, 2.65 mM NaH2PO4, 10 mM Tris-HCl, 100 mM NaCl, 20 mM β-mercaptoethanol; pH=8.0) and incubated for 2 hours at 37° C. and 150 rpm. After centrifugation at 35300 g, the supernatant containing the denatured and solubilized protein was collected.

The His6-tagged fusion protein was purified by immobilized metal ion affinity chromatography (IMAC) on columns with 1 ml Talon® metal (Co2+) affinity resin (BD Biosciences Clontech) using an automated protein purification procedure (Steen Jet at (2006) Protein Expr. Purif. 46:173-178) on an ASPEC XL4™ (Gilson). The resin was equilibrated with 20 ml denaturing washing buffer (6 M guanidine hydrochloride, 46.6 mM Na2HPO4, 3.4 mM NaH2PO4, 300 mM NaCl, pH 8.0-8.2). Clarified cell lysates were then added to the column. Thereafter, the resin was washed with a minimum of 31.5 ml washing buffer prior to elution in 2.5 ml elution buffer (6 M urea, 50 mM NaH2PO4, 100 mM NaCl, 30 mM acetic acid, 70 mM Na-acetate, pH 5.0). The eluted material was fractioned in three pools of 500, 700 and 1300 μl. The 700 μl fraction, containing the antigen, and the pooled 500 and 1300 μl fractions were stored for further use.

The antigen fraction was diluted to a final concentration of 1 M urea with phosphate buffered saline (PBS; 1.9 mM NaH2PO4, 8.1 mM Na2HPO4, 154 mM NaCl) followed by a concentration step to increase the protein concentration using Vivapore 10/20 ml concentrator with molecular weight cut off at 7500 Da (Vivascience AG). The protein concentration was determined using a bicinchoninic acid (BCA) micro assay protocol (Pierce) with a bovine serum albumin standard according to the manufacturer's recommendations. The protein quality was analyzed on a Bioanalyzer instrument using the Protein 50 or 200 assay (Agilent Technologies).

b) Results

A gene fragment corresponding to nucleotides 1058-1438 of the long transcript (SEQ ID NO:3) of the HER2 gene and encoding a peptide (SEQ ID NO:1) consisting of amino acid residues 274-400 of the target protein HER2 (SEQ ID NO:2) was successfully isolated by RT-PCR from a human RNA pool using primers specific for the protein fragment.

A clone encoding the correct amino acid sequence was identified, and, upon expression in E. coli, a single protein of the correct size was produced and subsequently purified using immobilized metal ion chromatography. After dilution of the eluted sample to a final concentration of 1 M urea and concentration of the sample to 1 ml, the concentration of the protein fragment was determined to be 8.6 mg/ml and was 99.5% pure according to purity analysis.

### 2. Generation of Antibodies

a) Materials and Methods

The purified HER2 fragment as obtained above was used as antigen to immunize a rabbit in accordance with the national guidelines (Swedish permit no. A 84-02). The rabbit was immunized intramuscularly with 200 μg of antigen in Freund's complete adjuvant as the primary immunization, and boosted three times in four weeks intervals with 100 μg antigen in Freund's incomplete adjuvant.

Antiserum from the immunized animal was purified by a three-step immunoaffinity based protocol (Agaton C et al (2004) J. Chromatogr. A 1043:33-40; Nilsson P et al (2005) Proteomics 5:4327-4337). In the first step, 7 ml of total antiserum was buffered with 10×PBS to a final concentration of 1×PBS (1.9 mM NaH2PO4, 8.1 mM Na2HPO4, 154 mM NaCl), filtered using a 0.45 μm pore-size filter (Acrodisc®, Life Science) and applied to an affinity column containing 5 ml N-hydroxysuccinimide-activated Sepharose™ 4 Fast Flow (GE Healthcare) coupled to the dual affinity tag protein His6-ABP (a hexahistidyl tag and an albumin binding protein tag) expressed from the pAff8c vector and purified in the same way as described above for the antigen protein fragment. In the second step, the flow-through, depleted of antibodies against the dual affinity tag His6-ABP, was loaded at a flow rate of 0.5 ml/min on a 1 ml Hi-Trap NHS-activated HP column (GE Healthcare) coupled with the HER2 protein fragment used as antigen for immunization (SEQ ID NO:1). The His6-ABP protein and the protein fragment antigen were coupled to the NHS activated matrix as recommended by the manufacturer. Unbound material was washed away with 1×PBST (1×PBS, 0.1% Tween20, pH 7.25), and captured antibodies were eluted using a low pH glycine buffer (0.2 M glycine, 1 mM EGTA, pH 2.5). The eluted antibody fraction was collected automatically, immediately after elution, relevant fractions were pooled and pH adjusted to 7.25 using 1M Tris-HCl and 10×PBS. The pooled fraction was denoted msAb-866.

b) Results

See section 3b below.

### 3. Generation of Region Specific Antibodies

a) Materials and Methods

Three fragments of the target protein encoded by the EnsEMBL Gene ID ENSG00000141736 (EnsEMBL, www.ensembl.org) were selected for suitable overlaps with the protein fragment used for immunization (SEQ ID NO:1). These three protein fragments, corresponding to amino acid residues 236-363 (SEQ ID NO:4), 347-492 (SEQ ID NO:5) and 364-530 (SEQ ID NO:6) of the HER2 protein (SEQ ID NO:2; EnsEMBL entry no. ENSP00000269571), were produced in analogous way to the protein fragment used for immunization (SEQ ID NO:1) as described above.

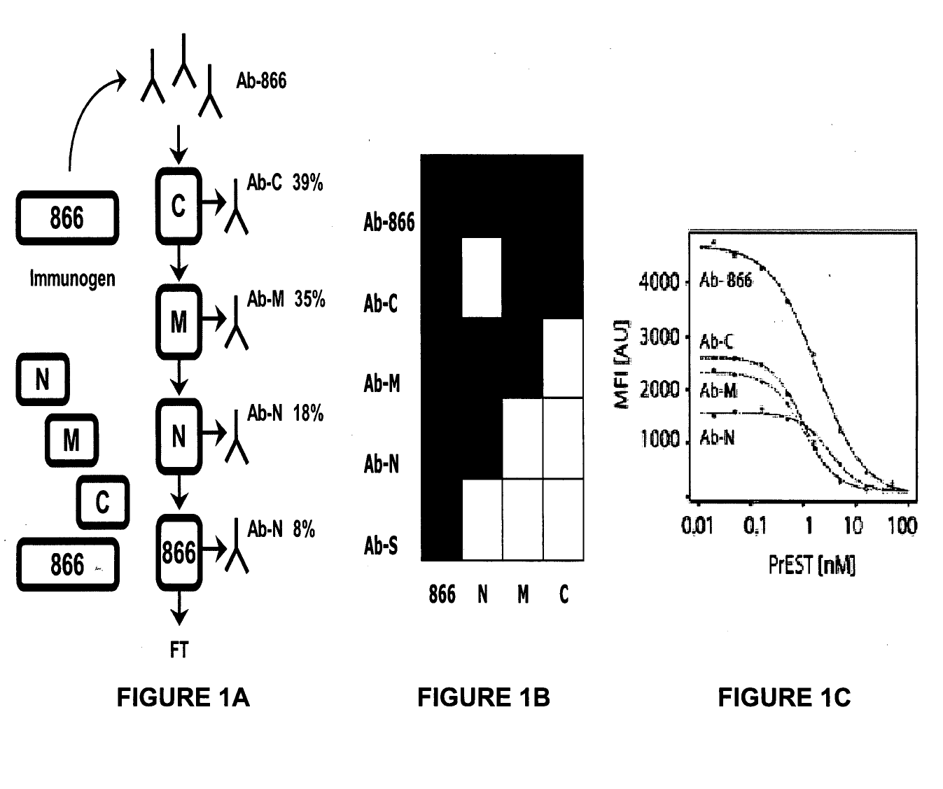

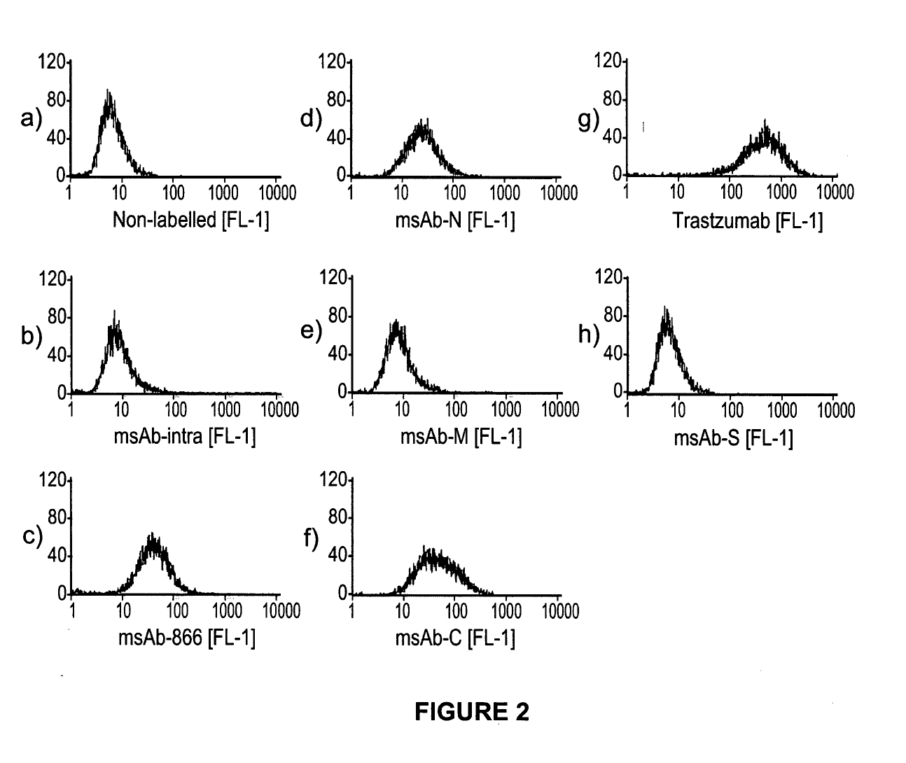

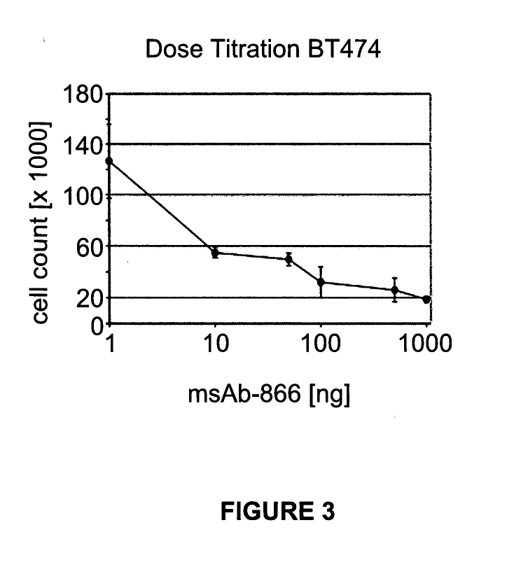

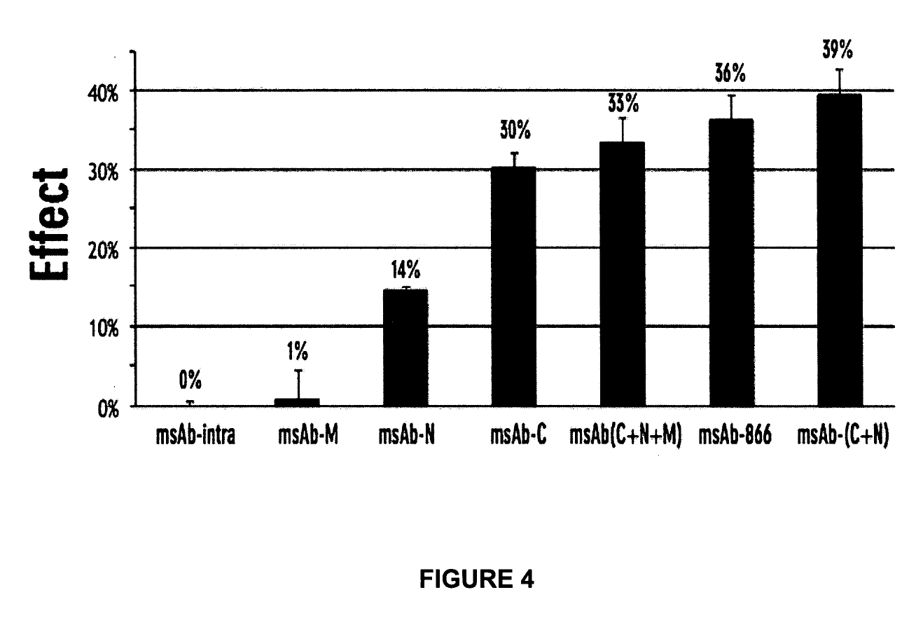

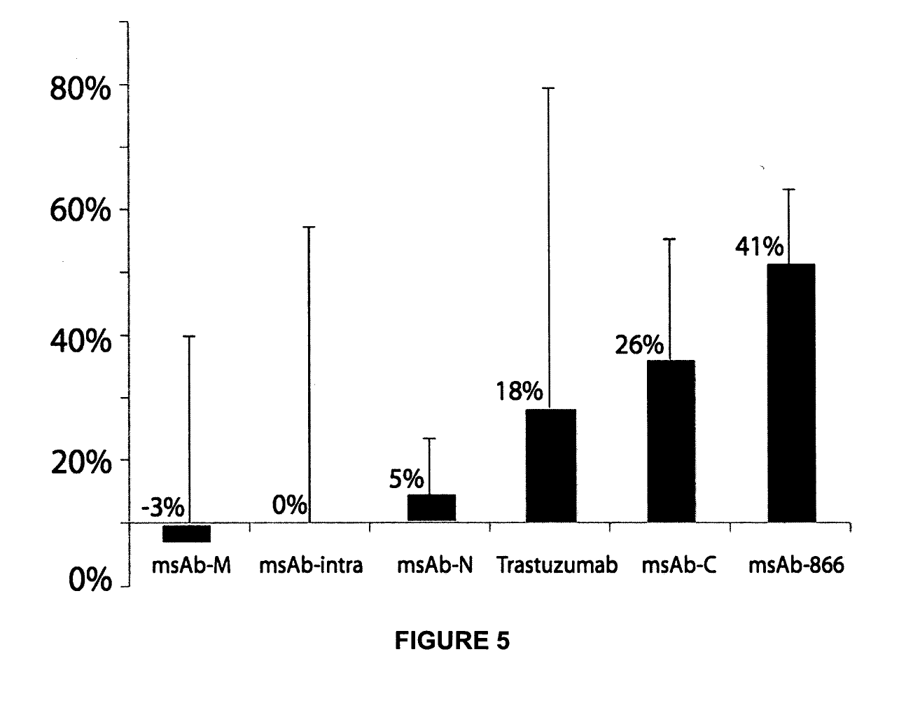

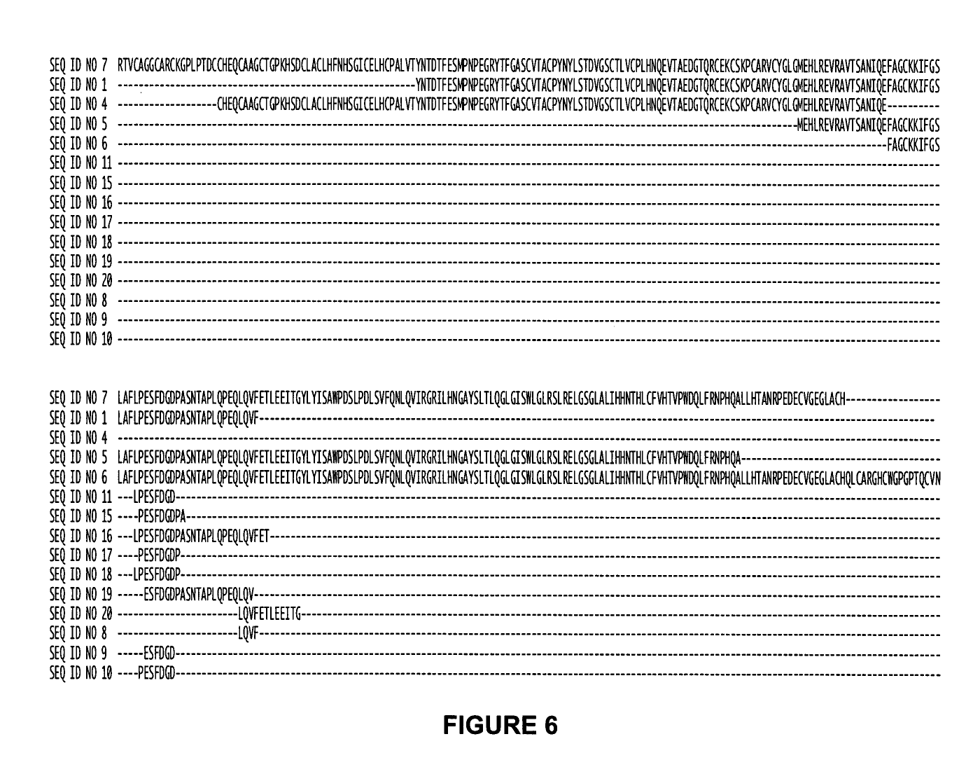

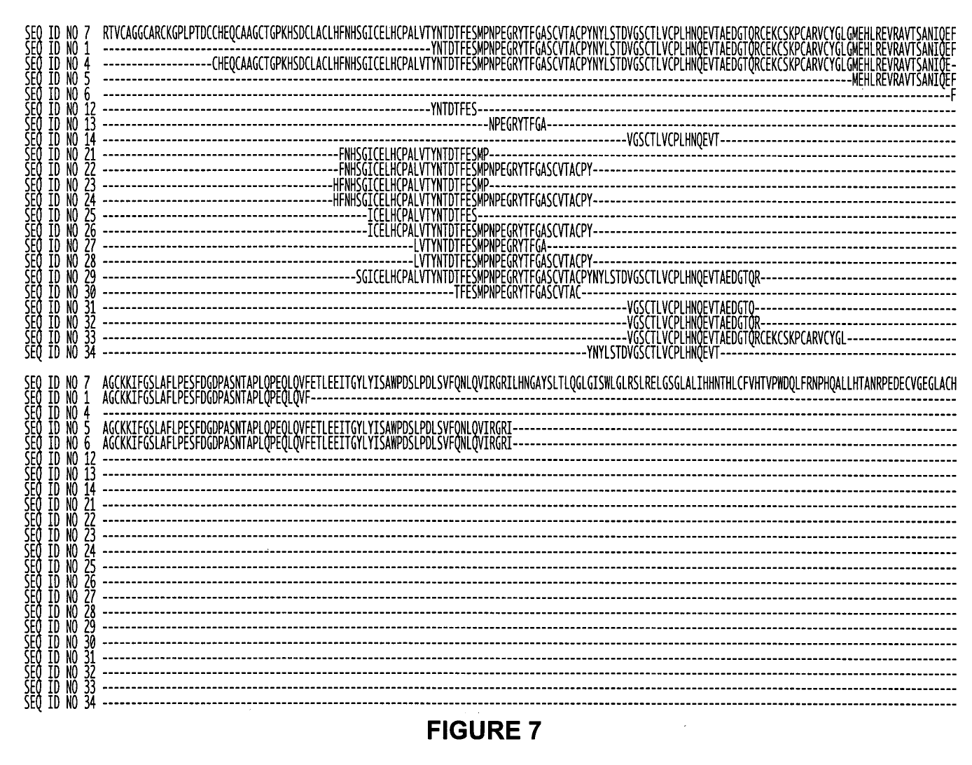

10 ml raw serum from the same immunization as described in 2a) was buffered in PBS, sterile filtered and depleted from Tag-specific His6-ABP antibodies using same protocol as above (Larsson et al., 2006). Depleted flow-through antibodies were affinity purified using an Äkta Explorer (GE Health Care AB) system with four serially connected affinity 1 ml HiTrap columns in the following order, C-terminal (SEQ ID NO:6), middle (SEQ ID NO:5), N-terminal (SEQ ID NO:4) and full antigen column (SEQ ID NO:1) (FIG. 1a). Antibodies were loaded on to the columns at a speed of 0.5 ml/min and unbound material was washed away with 20 column volumes of washing buffer. Bound antibodies were fractionated into 250 ul fractions after separate elution under low pH (Larsson et al., 2006). Immediately after elution relevant fractions were pooled and pH adjusted to 7.25 using 1M Tris-HCl and 10×PBS. No glycerol or NaN3 was added in order to not interfere with subsequent studies. The pooled fractions from respective column were denoted respectively: antibodies eluted from C-terminal column (SEQ ID NO:6) were denoted msAb-C, antibodies eluted from middle column (SEQ ID NO:5) were denoted msAb-M, antibodies eluted from, N-terminal (SEQ ID NO:4) were denoted msAb-N and antibodies eluted from full antigen column (SEQ ID NO:1) were denoted msAb-S.

b) Results

In brief four affinity columns with specific protein sequence corresponding to the C-terminal, middle and N-terminal part, respectively, of the protein fragment used for immunization as well as the full antigen fragment (SEQ ID NO:1) were serially coupled to enable selective affinity chromatography. The method enabled separation of the antigen specific antibodies to be split into four distinct antibody populations: msAb-N (18% of the population), msAb-M (35% of the population), msAb-C (39% of the population), msAb-S (8% of the population) (FIG. 1a).

### 4. Validation of Affinity Purification

a) Protein Arrays

The specificity and selectivity of the affinity purified antibody fraction were analyzed by binding analysis against the antigen itself, including the protein fragment used for immunization (SEQ ID NO:1) and protein fragments overlapping it (SEQ ID NO:4-6), and against 92 other human protein fragments in a protein array set-up (Nilsson P et al (2005) Proteomics 5:4327-4337). The protein fragments were diluted to 40 μg/ml in 0.1 M urea and 1×PBS (pH 7.4) and 50 μl of each were transferred to the wells of a 96-well spotting plate. The protein fragments were spotted in duplicate and immobilized onto epoxy slides (SuperEpoxy, TeleChem) using a pin-and-ring arrayer (Affymetrix 427). The slide was washed in 1×PBS (5 min) and the surface was then blocked (SuperBlock®, Pierce) for 30 minutes. An adhesive 16-well silicone mask (Schleicher & Schuell) was applied to the glass before the mono-specific antibodies were added (diluted 1:5000 in 1×PBST to appr. 50 ng/ml) and incubated on a shaker for 60 min. Affinity tag-specific IgY antibodies were co-incubated with the mono-specific antibodies in order to quantify the amount of protein in each spot. The slide was washed with 1×PBST and 1×PBS twice for 10 min each. Secondary antibodies (goat anti-rabbit antibody conjugated with Alexa 647 and goat anti-chicken antibody conjugated with Alexa 555, Molecular Probes) were diluted 1:60000 to 30 ng/ml in 1×PBST and incubated for 60 min. After the same washing procedure, as for the first incubation, the slide was spun dry and scanned (G2565BA array scanner, Agilent); thereafter images were quantified using image analysis software (GenePix 5.1, Axon Instruments).

b) Suspension Bead Arrays

In addition, specificity, selectivity and relative affinities were analyzed using a Luminex suspension bead array system. Multiplexed analysis of binding specificities was performed as previously described (Schwenk et al., 2007). In short, antibody dilutions and a bead mixture of 100 bead IDs corresponding to 98 protein fragments, including the antigen used for immunization (SEQ ID NO:1) and the three fragments used for sub-purification antigens (SEQ ID NO:4-6), one HisABP fragment and one anti-rabbit IgG antibody were prepared in PBST. 45 μl of msAb dilutions were added to 5 μl of beads and incubated for 60 min under constant mixing in a 96 well plate (Corning). Subsequently, 25 μl of R-Phycoerythrin labeled anti-rabbit IgG antibody (0.5 μg/ml, Jackson ImmunoResearch) or were added for a final incubation of 60 min.

c) Multiplexed Competition Assays

Serial dilutions of competitor protein fragments were prepared in PBST and mixed at a 1:1 ratio with solutions of msAb-866, msAb-N, msAb-M and msAb-C. Incubation took place in a total volume of 50 μl for 60 min under permanent mixing. Subsequently, the msAb-PrEST solutions were transferred to a second plate containing 5 μl of bead mixtures per well. After 60 min, 25 μl of R-Phycoerythrin labeled anti-rabbit IgG antibody (0.5 μg/ml, Jackson ImmunoResearch) were added and incubated for another 60 min. Three independent replicates were performed and average values of those were used for data analysis. A four parameter logistical function was chosen for fitting competition curves to calculate EC50 values and to compare relative binding qualities. As a measure for competition, resulting curves were observed upon their shape and their estimated EC50 values that had to be of a greater number value than the standard error.

d) Suspension Array Read-Out and Data Analysis

Measurements were performed using Luminex LX200 instrumentation with Luminex IS 2.3 software. For each experiment 100 events per bead ID were counted and the median fluorescence intensity (MFI) was chosen to display interactions. Data analysis and graphical representations were performed with R, a language and environment for statistical computing and graphics (Ihaka and Gentleman, 1996).

e) Results

To validate antibody specificity and selectivity after affinity purification, a protein microarray analysis was performed using both planar arrays and Luminex suspension bead array technology. The analysis validated a successful depletion of antibodies directed against His6-tag and ABP-tag (results not shown) as well as ensured highly specific antibodies possessing low potential unspecific interactions with other protein fragments immobilized (FIG. 1b).

To quantify the amount of protein in each spot of the protein array, a two-color dye labeling system was used, with a combination of primary and secondary antibodies. Tag-specific IgY antibodies generated in hen were detected with a secondary goat anti-hen antibody labeled with Alexa 555 fluorescent dye. The specific binding of the rabbit msAb to its antigen on the array was detected with a fluorescently Alexa 647 labeled goat anti-rabbit antibody. Each protein fragment was spotted in duplicates. The protein array analysis using planar and suspension bead array showed that the affinity purified mono-specific antibodies; msAb-866, msAb-N, msAb-M, msAb-C, ms-Ab-S against HER2, were highly selective to the correct protein fragments and have a very low background to all other protein fragments analyzed. In addition relative affinities were determined using a competition assay, where the interaction between mono-specific antibody and immobilized full antigen (SEQ ID NO:1) protein fragment on bead is challenged using an increasing concentration of soluble full antigen (SEQ ID NO:1) competitor protein fragment (FIG. 1c, table 1). An apparent affinity in the low nanomolar range was determined for antibodies; msAb-866 (1.9 nM), msAb-N (4.5 nM), msAb-M (0.7 nM) and msAb-C (1.2 nM), when their interaction to beads coupled to protein fragment used in immunization (SEQ ID NO:1) was challenged with soluble protein fragment (SEQ ID NO:1).

TABLE 1
Relative affinities

| Protein fragment | EC50 |
|---|---|
| Ab-866 | 1.9 nM |
| Ab-C | 1.2 nM |
| Ab-M | 0.7 nM |
| Ab-N | 4.5 nM |

### 5. Cell Study

a) Cell Culture

BT474 breast cancer cells were purchased from the American Type Culture Collection (ATCC, Manassas, Va.) and maintained in RPMI supplemented with 10% FCS and 1% Bovine Insulin and kept in 37° C. at 5% CO2 humidified atmosphere.

b) Cell Binding Assay

Cells were released from culture dish by trypsination, centrifuged and resuspended in PBS:HSA (PBS pH 7.2 supplemented with 1% Human Serum Albumin) and counted. 150000 cells were labelled for 45 min with 0.35 microgram antibody (msAb-N, msAb-M, msAb-C and msAb-866) in a reaction volume of 75 microlitre in a 96-wellplate in room temperature. Unbound antibodies was washed away using 2×100 microlitre PBS:HSA as washing agent. This was followed by antibody labelling using 0.35 microgram secondary goat anti-rabbit monoclonal antibody (Invitrogen) conjugated to Alexa 488 in a reaction volume of 75 microlitre for 45 min at room temperature. Cells were washed in PBS:HSA 2×100 microlitre and resuspended in a sample tube to a final volume of 150 ul. Ability for antibodies to bind BT474 cells was evaluated by fluorescence activated cellsorting using a BD FACS Vantage SE flowcytometer (BD Biosciences) measuring fluorescence emission at FL-1 (excitation at 488 nm). Equimolar amounts of rabbit mono-specific antibody (msAb-intra) targeting the intracellular part of HER2 (HPA001383, Atlas Antibodies AB, Sweden) was used as negative control along with cells labelled only with secondary antibodies. Trastuzumab (Herceptin, Roche) was used as positive cell labeling control using Alexa 488 goat anti-human monoclonal antibodies (Invitrogen) as secondary reagent.

c) Dose-Response Studies

BT474 cells were seeded at 5×10⁴ cells/well in 24-well dishes. After 24 h, cells were treated in triplicate dilutions of msAb-866 in concentrations ranging from 1 ng/ml to 1000 ng/ml. Cells treated with PBS pH 7.2 was used as control. After 5 days, cells were trypsinized and counted three times each. Growth inhibition was calculated as percentage of cells compared with untreated cultures.

d) First growth inhibition study

BT474 cells were seeded at 5×10⁴ cells/well in 24-well dishes at day 0. Two reconstitution mixes of antibodies were made using fraction ratios obtained in 3b): msAb-N and msAb-C were mixed at 18:39 ratio denoted msAb-NC; and msAb-N, msAb-M and msAb-C were mixed at a 18:35:39 ratio denoted msAb-NMC. After 24 h, cells were treated in triplicate dilutions of msAb-866, msAb-N, msAb-M, msAb-C, msAb-NC and msAb-NMC using a final antibody concentration of 500 ng/ml. msAb-intra and PBS pH 7.2 were used as controls. After 5 days, cells were trypsinized and counted three times each. Growth inhibition was calculated as percentage of cells as compared to cultures treated with the control antibody msAb-intra.

e) Second Growth Inhibition Study

BT474 cells were seeded at day 1 in triplicates at 5×10⁴ cells/well together with dilutions of msAb-866, msAb-C, and Trastuzumab using a final antibody concentration of 500 ng/ml. msAb-intra and PBS pH 7.2 were used as controls. After 4 days, cells were trypsinized and counted three times each. Growth inhibition was calculated as percentage of cells as compared to cultures treated with the control antibody msAb-intra.

f) Results

The Fluorescence Activated Cell Sorting of BT474 cells labeled with msAb-866, msAb-N and msAb-C showed an enriched population with higher fluorescence over unlabeled cells, indicating cell binding whereas no significant fluorescence enrichment was seen for msAb-intra or msAb-M (FIG. 2).

A critical antibody concentration of 250-500 ng/ml for significant influence on cell growth was seen at day 5 in the dose-response study treating BT474 cells with msAb-866 (FIG. 3).

In the first growth inhibition study of BT474 cells using 500 ng/ml of respective antibody, msAb-M showed 1% growth inhibition effect, msAb-N showed 14% growth inhibition effect, msAb-C showed 30% growth inhibition effect, msAb-NMC showed 33% growth inhibition effect, msAb-866 showed 36% growth inhibition effect, msAb-NC showed 39% growth inhibition effect (FIG. 4).

Consequently, both Ab-N and Ab-C taken alone showed a substantial effect. Combinations comprising Ab-N and Ab-C also showed a substantial effect. Further, it has been noted by the inventors that all the antibody samples that showed a high effect (≧30%) comprised Ab-C.

Also, the samples containing both Ab-C and Ab-N generally showed a higher effect as compared to either Ab-C or Ab-N taken alone.

The highest effect was observed for a sample containing Ab-C and Ab-N.

In the second growth inhibition study of BT474 cells at day 4 using 500 ng/ml of respective antibody, Ab-866 showed about 41%, Ab-C about 26%, trastuzumab about 18% and Ab-N about 5% growth inhibition effect relative to msAb-intra (FIG. 5).

Further, it has been noted by the inventors that Ab-C and Ab-866 in this setup showed a higher effect than trastuzumab (Herceptin), which is an approved therapeutic antibody targeting the extracellular domain of HER2. This indicates that an antibody targeting the HER2 subset of amino acid residues 1-37 of SEQ ID NO:6, or a composition comprising antibodies targeting the HER2 subset of amino acid residues 1-37 of SEQ ID NO:6 and the HER2 subset of amino acid residues 39-111 of SEQ ID NO:4, respectively, may be used for treatment of disorders characterized by the overexpression of HER2.

The highest effect was observed for Ab-866, which contains both Ab-C and Ab-N.

### 6. Epitope Mapping Using Bacterial Display

a) Subcloning of libraries into the staphylococcal display vector The E. coli strain RR10M15 (Rather, U. pUR 250 allows rapid chemical sequencing of both DNA strands of its inserts. Nucleic. Acids Res. 10, 5765-5772 (1982)) was used as host strain for plasmid constructions. A new staphylococcal vector, pSCEM1, was created by ligating a gene fragment containing a new restriction site (PmeI) to the previously described staphylococcal vector pSCXm (Wernérus, H. & Ståhl, S. Vector engineering to improve a staphylococcal surface display system. FEMS Microbiol Lett 212, 47-54 (2002)) digested with BamHI and SalI (New England Biolabs, Beverly, Mass.). Template for amplification of HER2-ECD with N-terminal FLAG sequence was obtained. The gene-fragment was amplified by PCR (9.6 ml, pooled) and sonicated (21% amplitude, constant sonication) using a microtip for 60 min in a 50 ml Falcon tube on ice in order to generate random fragments. Samples were thereafter concentrated by ultrafiltration using Centricon Plus 20 column (CO 10 kDa; Millipore, Billerica, Mass.). Concentrated fragments were blunt-ended and phosphorylated by addition of T4 DNA polymerase and T4 polynucleotide kinase (New England Biolabs) according to the supplier's recommendations. The blunt-ended gene fragments were thereafter ligated using T4 DNA Ligase (Invitrogen, Carlsbad, Calif.) into the staphylococcal display vector, pSCEM1, digested with PmeI (New England Biolabs). The library was transformed to electrocompetent S. carnosus TM300 (Götz, F. Staphylococcus carnosus: a new host organism for gene cloning and protein production. Soc. Appl. Bacteriol. Symp. Ser. 19, 49S-53S (1990)) as described previously (Löfblom, J., Kronqvist, N., Uhlén, M., Ståhl, S. & Wernérus, H. Optimization of electroporation-mediated transformation: Staphylococcus carnosus as model organism. J Appl Microbiol 102, 736-747 (2007)) and stored in 15% glycerol at −80° C.

b) Cell labeling and fluorescence-activated cell sorting (FACS) An aliquot of Sc:HER2-lib or Sc:Ephrin-B3-lib (at least ten times the library size) was inoculated to 100 ml TSB+Y (Tryptic soy broth+yeast extract) with 20 μg ml-1 chloramphenicol and grown over night at 37° C. and 150 rpm. After 16 hours, 10⁷ cells were washed with 1 ml phosphate-buffered saline (PBS, pH 7.4) with 0.1% Pluronic® F108 NF Surfactant (PBSP; BASF Corporation, Mount Olive, N.J.). The cells were pelleted by centrifugation (3500×g, 4° C., 6 min) and resuspended in 100 μl PBSP containing antibody (i.e. the antibody used for epitope mapping; typically at a concentration around 100 nM) and incubated at room temperature with gentle mixing for 1 hour to reach equilibrium binding. The cells were thereafter washed with 1 ml of ice-cold PBSP followed by incubation in 1 ml PBSP containing 4 μg ml-1 Alexa Fluor® 488 goat anti-rabbit IgG or 4 μg ml-1 Alexa Fluor® 488 goat anti-mouse IgG (Invitrogen) and 225 nM Alexa Fluor® 647 HSA conjugate for 1 hour on ice in the dark. After a final washing step in 1 ml of ice-cold PBSP, the cells were resuspended in 300 μl of ice-cold PBSP before sorting. Cells were sorted using a FACSVantage SE (BD Biosciences, San Jose, Calif.) flow cytometer. The cells were sorted directly into 0.5 ml B2 medium (Löfblom, J., Kronqvist, N., Uhlén, M., Ståhl, S. & Wernérus, H. Optimization of electroporation-mediated transformation: Staphylococcus carnosus as model organism. J Appl Microbiol 102, 736-747 (2007)) and spread onto blood agar base (Merck) plates containing 10 μg ml-1 chloramphenicol and incubated at 37° C. for 24 hours. In the last round, cells were sorted into individual wells in 96-well plates, containing semi-solid medium, to form colonies.

c) DNA Sequencing and BLAST Alignment

Parts of each colony were transferred to two separate wells in 96-well plates for PCR. The insert region of the staphylococcal display vector was amplified by PCR using two distinct primer pairs, yielding two PCR products containing a biotin molecule in the forward end and in the reverse end, respectively. A 10 cycles Pyrosequencing at both ends of each insert was performed according to manufacturer's instructions using a PSQ™ 96 HS instrument (Biotage AB, Uppsala, Sweden). Epitope sequences were mapped to the antigen sequence using BLAST (Altschul et al, Basic local alignment search tool, J. Mol. Biol. 147:195-197, (1990)).

d) Results

DNA of the extra cellular domain of HER2 (aa 27-653 of ENSP00000269571 or by 317-2196 ENST00000269571) was amplified by PCR using vector pAY593 as template. The amplified DNA was fragmentized to various lengths (approximately 50-350 bp) by sonication, followed by ligation into the staphylococcal display vector (pSCEM1) and transformed into S. Carnosus yielding around 30000 transformants. In-frame DNA fragments were displayed as peptides on the staphylococcal surface. After incubation with antibody and fluorescently labeled secondary reagents, positive and negative cells were separately sorted using flow cytometry in order to isolate epitope and non-epitope presenting cells. Isolated cells were sequenced by pyrosequencing and sequences finally aligned to the HER2 antigen for identification of epitopes.

A dual-labeling strategy with real-time monitoring of the surface expression level was used (Löfblom, J., Wernérus, H. & Ståhl, S. Fine affinity discrimination by normalized fluorescence activated cell sorting in staphylococcal surface display. FEMS Microbiol Lett 248, 189-198 (2005)). It allowed for normalization of the binding signal with the expression level, provided low cell-to-cell variations and made discrimination of different epitope populations possible. Further, it also allowed for a parallel assay to determine non-binding peptides displayed on the surface.

Four epitopes (SEQ ID NO:8 and SEQ ID NO:12-14) specific for msAb-866 were confirmed. A second round of epitope mapping was undertaken for msAb-C revealing one additional epitope (SEQ ID NO:11). The epitopes SEQ ID NO:8 and SEQ ID NO:11, including the variants of the latter SEQ ID NO:9-10, are located on the C-fragment whereas the epitopes SEQ ID NO:12-14 are located on the N-fragment.

The interactions of e.g., fragments SEQ ID NO:11 and 15-20 were utilized in the establishment of the epitopes SEQ ID NO:8 and SEQ ID NO:11, including it's variants SEQ ID NO:9-10 (FIG. 6).

Further, the interactions of e.g., fragments SEQ ID NO:21-34 were utilized in the establishment of the epitopes SEQ ID NO:12-14 (FIG. 7).

All cited material, including but not limited to publications, DNA or protein data entries, and patents, referred to in this application are herein incorporated by reference.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### 7. Generation of Monoclonal Antibodies

Monoclonal antibodies may be produced based on the hybridoma technology developed by Kohler and Milstein (Kohler, G and Milstein, C, 1973, Nature 256, 495-497). The inventors give here a brief description on how to develop monoclonal antibodies against HER2 epitopes according to the present disclosure. The monoclonal antibody should be capable of selective interaction with amino acid sequences LQVF (SEQ ID NO:8) or ESFDGD (SEQ ID NO:9). SEQ ID NO: 1 may be used as the antigen and its production is explained in Examples, section 1. An alternative approach is to synthesize a peptide including the inventive epitopes, e.g. a peptide consisting of the amino acid sequence CAFLPESFDGDPASNTAPLQPEQLQVFET, and use this peptide as the antigen.

Antigen is injected subcutaneously into BALB/c mice (4-6 weeks old, female) at three-week intervals. Prior to immunization the antigen is mixed with complete Freund's adjuvant for the first injection and incomplete Freund's adjuvant for the following injections. Three days before fusion, the mouse is last challenged with antigen intravenously.

Hybridomas are generated by fusion of splenocytes from the immunized mice with a Sp2/0 myeloma cell line. Then, several hybridoma cell lines are screened using ELISA, and cell lines that secrete antibodies specific for one or more fragment(s) comprising LQVF (SEQ ID NO:8) and/or ESFDGD (SEQ ID NO:9) are identified and selected for further characterization.

As some aspects of the present disclosure involve use of the HER2 antibody as a therapeutic agent, further characterization involves testing of antibodies in cell supernatants from the selected hybridoma cell lines in cell binding assays as presented in Example section 5b. Cell lines with antibodies that bind to the HER2 receptor expressed on the surface of BT474 cells are selected for subcloning and expansion.

Further characterization may include growth inhibition studies in line with the results presented in Example section 5d-5e, to confirm that the monoclonal antibodies exhibit the therapeutically interesting growth inhibition effect. Finally, epitope mapping as presented in Example section 6a-d may be done, to confirm that the monoclonal antibodies from the selected cell lines interact with the expected eptiope(s).

Prior to introduction of the monoclonal antibody as a therapeutic agent, its immunogenicity may be reduced. Murine monoclonal antibodies may be engineered to become chimeric or humanized, thereby removing at least part of their immunogenic content and increasing their immunologic efficiency. Alternatively, fully human monoclonal antibodies may be produced using transgenic mice or phage display libraries.

1.-86. (canceled)

87. Affinity ligand capable of selective interaction with a subset consisting of 37 consecutive amino acid residues or less from extracellular domains 2 and 3 of Human Epidermal growth factor Receptor 2 (HER2) (SEQ ID NO:7), said subset comprising the amino acid sequence LQVF (SEQ ID NO:8) and/or ESFDGD (SEQ ID NO:9).

88. Affinity ligand according to claim 87, wherein said subset consists of 26 amino acid residues or less.

89. Affinity ligand according to claim 88, wherein said subset consists of an amino acid sequence selected from the group consisting of SEQ ID NO:11 and 15-20.

90. Affinity ligand according to claim 88, wherein said subset consists of 21 amino acid residues or less.

91. Affinity ligand according to claim 87, wherein said subset comprises LQVF (SEQ ID NO:8) and/or LPESFDGD (SEQ ID NO:11).

92. Affinity ligand according to claim 87, wherein said subset consists of the sequence of amino acid residues 1-37 of SEQ ID NO:6.

93. Affinity ligand according to claim 87, which inhibits growth of human breast cancer cells in culture by 20-100%.

94. Affinity ligand according to claim 93, wherein the human breast cancer cells are BT474 breast cancer cells.

95. Affinity ligand according to claim 93, which inhibits growth at a concentration of 500 ng/ml.

96. Affinity ligand according to claim 87, which binds the subset with an EC50 of less than 100 nM.

97. Affinity ligand according to claim 87, which is an antibody or a fragment or derivative thereof.

98. Affinity ligand according to claim 97, which is a monoclonal antibody.

99. Affinity ligand according to claim 97, which is a chimeric or humanized monoclonal antibody.

100. A composition comprising an affinity ligand according to claim 87 and a second affinity ligand capable of selective interaction with a second subset of 73 consecutive amino acid residues or less from extracellular domains 2 and 3 of Human Epidermal growth factor Receptor 2 (HER2) (SEQ ID NO:7), said second subset comprising one or more amino acid sequences selected from the group consisting of SEQ ID NO:12, SEQ ID NO:13 and SEQ ID NO:14.

101. Composition comprising an affinity ligand according to claim 87 and a tyrosine kinase inhibitor against Human Epidermal growth factor Receptor 2 (HER2).

102. Method of preparation of a therapeutic antibody for the treatment of a disorder characterized by the overexpression of Human Epidermal growth factor Receptor 2 (HER2), comprising an immunization using a polypeptide which consists of 37 consecutive amino acid residues or less from extracellular domains 2 and 3 of HER2 (SEQ ID NO:7) and comprises the amino acid sequence LQVF (SEQ ID NO:8) and/or ESFDGD (SEQ ID NO:9) as an antigen.

103. Method according to claim 102, wherein the therapeutic antibody is monoclonal.

104. Method according to claim 102, wherein the therapeutic antibody is chimeric or humanized.

105. Method of selection or purification of a therapeutic antibody for the treatment of a disorder characterized by the overexpression of Human Epidermal growth factor Receptor 2 (HER2), comprising the use of a polypeptide which consists of 37 consecutive amino acid residues or less from extracellular domains 2 and 3 of HER2 (SEQ ID NO:7) and comprises the amino acid sequence LQVF (SEQ ID NO:8) and/or ESFDGD (SEQ ID NO:9).

106. Method for identification of an affinity ligand for treatment of a disorder characterized by the overexpression of Human Epidermal growth factor Receptor 2 (HER2), comprising the steps of:
a) contacting a polypeptide comprising a subset according to claim 87 with a putative affinity ligand in conditions that enable binding; and
b) determining whether the putative affinity ligand binds to the subset.

107. Method according to claim 106, wherein the polypeptide of step a) consists of a subset consisting of 37 consecutive amino acid residues or less from extracellular domains 2 and 3 of HER2 (SEQ ID NO:7) and comprising the amino acid sequence LQVF (SEQ ID NO:8) and/or ESFDGD (SEQ ID NO:9).

108. Method according to claim 106, wherein the disorder is a cancer, further comprising the step:
c) determining whether the putative affinity ligand inhibits growth or induces apoptosis of cancer cells, such as BT474 breast cancer cells.

109. Method of producing an affinity ligand, comprising:
identifying an affinity ligand using the method according of claim 106; and
producing said identified affinity ligand.

110. Method for producing a clone comprising the steps of:
a) providing cells obtained from a mammal which has been immunized with an antigen comprising a subset consisting of 37 consecutive amino acid residues or less from extracellular domains 2 and 3 of HER2 (SEQ ID NO:7) and comprising the amino acid sequence LQVF (SEQ ID NO:8) and/or ESFDGD (SEQ ID NO:9), which cells comprise DNA encoding an antibody capable of selective interaction with the subset; and
b) fusing said cells with myeloma cells to obtain at least one clone.

111. Method according to claim 110, further comprising the step of:
a′) immunizing the mammal with the antigen,
wherein step a′) precedes step a).

112. Method according to claim 110, further comprising the step of:
c) selecting a clone from step b) which expresses antibodies capable of selective interaction with the subset.

113. Method according to claim 110, wherein the antigen of step a) consists of 37 consecutive amino acid residues or less from extracellular domains 2 and 3 of HER2 (SEQ ID NO:7) and comprises the amino acid sequence LQVF (SEQ ID NO:8) and/or ESFDGD (SEQ ID NO:9).

114. Method according to claim 110 or 112, further comprising the step:
d) providing a clone obtained in step b) or selected in step c), and merging
DNA from the clone, which DNA encodes at least the part of an antibody expressed by the clone that selectively interacts with the subset, with human antibody encoding DNA; and
e) incorporating the merged DNA from step d) in cells to obtain a clone for expression of a therapeutic antibody for treatment of a disorder characterized by the overexpression of Human Epidermal growth factor Receptor 2 (HER2).

115. Method of producing an affinity ligand comprising: producing a clone using the method according to claim 110; and obtaining said affinity ligand from said clone.

116. Method of treatment of a mammalian subject having, or suspected of having, a disorder characterized by the overexpression of Human Epidermal growth factor Receptor 2 (HER2), comprising administering an effective amount of an affinity ligand according to claim 87 or a composition comprising said affinity ligand and a second affinity ligand capable of selective interaction with a second subset of 73 consecutive amino acid residues or less from extracellular domains 2 and 3 of HER2 (SEQ ID NO:7), said second subset comprising one or more amino acid sequences selected from the group consisting of SEQ ID NO:12, SEQ ID NO:13 and SEQ ID NO:14, to the subject.

117. Method according to claim 116, further comprising administering a tyrosine kinase inhibitor against HER2 to the subject.

118. Method according to claim 116, wherein said subject has been treated by a therapeutic antibody capable of selective interaction with HER2, such as the extracellular domain of HER2, which therapeutic antibody is different from the affinity ligand.

119. Method according to claim 118, wherein said disorder characterized by the overexpression of HER2 is a cancer and said cancer has developed resistance to the therapeutic antibody.

120. Method according to claim 116, wherein the disorder characterized by the overexpression of HER2 is a cancer.

121. Method according to claim 120, wherein the cancer is selected from the group consisting of breast cancer, squamos cell cancinoma, lung cancer, such as small cell or non-small cell lung cancer, pancreatic cancer, glioblastoma, cervical cancer, ovarian cancer, vulval cancer, liver cancer, hepatoma, colorectal cancer, such as colon cancer, endometrial carcinoma, salivary gland carcinoma, kidney cancer, thyroid cancer, Wilm's tumor, bladder cancer, endometrial cancer, renal cancer, head and neck cancer, gastric cancer, esophageal cancer and prostate cancer.

122. Method according to claim 121, wherein the cancer is metastatic breast cancer.

123. An article of manufacture, comprising a container, a composition within the container comprising an affinity ligand according to claim 87 and a label on or associated with the container that indicates that said composition can be used for treating a disorder characterized by the overexpression of Human Epidermal growth factor Receptor 2 (HER2).

124. Article of manufacture according to claim 123, wherein the container has a sterile access port.

125. Article of manufacture according to claim 123, wherein the container is an intravenous solution bag or a vial having a stopper pierceable by a hypodermic needle.

126. Article of manufacture according to claim 123, further comprising a second container comprising a pharmaceutically acceptable buffer.


SEQUENCE LISTING (34 sequences)

SEQ ID NO: 1 - protein, 127 aa, Homo sapiens
YNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVF

SEQ ID NO: 2 - protein, 1255 aa, Homo sapiens [sequence omitted]

SEQ ID NO: 3 - DNA, 4624 bp, Homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 4 - protein, 128 aa, Homo sapiens
CHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQE

SEQ ID NO: 5 - protein, 146 aa, Homo sapiens
MEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQA

SEQ ID NO: 6 - protein, 167 aa, Homo sapiens
FAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVN

SEQ ID NO: 7 - protein, 296 aa, Homo sapiens
RTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACH

SEQ ID NO: 8 - protein, 4 aa, Homo sapiens
LQVF

SEQ ID NO: 9 - protein, 6 aa, Homo sapiens
ESFDGD

SEQ ID NO: 10 - protein, 7 aa, Homo sapiens
PESFDGD

SEQ ID NO: 11 - protein, 8 aa, Homo sapiens
LPESFDGD

SEQ ID NO: 12 - protein, 8 aa, Homo sapiens
YNTDTFES

SEQ ID NO: 13 - protein, 10 aa, Homo sapiens
NPEGRYTFGA

SEQ ID NO: 14 - protein, 16 aa, Homo sapiens
VGSCTLVCPLHNQEVT

SEQ ID NO: 15 - protein, 9 aa, Homo sapiens
PESFDGDPA

SEQ ID NO: 16 - protein, 26 aa, Homo sapiens
LPESFDGDPASNTAPLQPEQLQVFET

SEQ ID NO: 17 - protein, 8 aa, Homo sapiens
PESFDGDP

SEQ ID NO: 18 - protein, 9 aa, Homo sapiens
LPESFDGDP

SEQ ID NO: 19 - protein, 21 aa, Homo sapiens
ESFDGDPASNTAPLQPEQLQV

SEQ ID NO: 20 - protein, 12 aa, Homo sapiens
LQVFETLEEITG

SEQ ID NO: 21 - protein, 26 aa, Homo sapiens
FNHSGICELHCPALVTYNTDTFESMP

SEQ ID NO: 22 - protein, 44 aa, Homo sapiens
FNHSGICELHCPALVTYNTDTFESMPNPEGRYTFGASCVTACPY

SEQ ID NO: 23 - protein, 27 aa, Homo sapiens
HFNHSGICELHCPALVTYNTDTFESMP

SEQ ID NO: 24 - protein, 45 aa, Homo sapiens
HFNHSGICELHCPALVTYNTDTFESMPNPEGRYTFGASCVTACPY

SEQ ID NO: 25 - protein, 19 aa, Homo sapiens
ICELHCPALVTYNTDTFES

SEQ ID NO: 26 - protein, 39 aa, Homo sapiens
ICELHCPALVTYNTDTFESMPNPEGRYTFGASCVTACPY

SEQ ID NO: 27 - protein, 23 aa, Homo sapiens
LVTYNTDTFESMPNPEGRYTFGA

SEQ ID NO: 28 - protein, 31 aa, Homo sapiens
LVTYNTDTFESMPNPEGRYTFGASCVTACPY

SEQ ID NO: 29 - protein, 70 aa, Homo sapiens
SGICELHCPALVTYNTDTFESMPNPEGRYTFGASCVTACPYNYLSTDVGSCTLVCPLHNQEVTAEDGTQR

SEQ ID NO: 30 - protein, 22 aa, Homo sapiens
TFESMPNPEGRYTFGASCVTAC

SEQ ID NO: 31 - protein, 22 aa, Homo sapiens
VGSCTLVCPLHNQEVTAEDGTQ

SEQ ID NO: 32 - protein, 23 aa, Homo sapiens
VGSCTLVCPLHNQEVTAEDGTQR

SEQ ID NO: 33 - protein, 38 aa, Homo sapiens
VGSCTLVCPLHNQEVTAEDGTQRCEKCSKPCARVCYGL

SEQ ID NO: 34 - protein, 23 aa, Homo sapiens
YNYLSTDVGSCTLVCPLHNQEVT